SEQUENCE LISTING (30 sequences)

SEQ ID NO: 1 - protein, 604 aa, Aspergillus clavatus
MRFPSIFTAVLFAASSALAAPVNTTTEDETAQIPAEAVIGYSDLEGDFDVAVLPFSNSTNNGLLFINTTIASIAAKEEGVSLEKRPAIPQLPVPPPFEIQESSLLSGNARFRQVRGNTTFDQLIDHDNPELGTFQQRFWWSSEFWKGPGSPVVLFTPGEADAPGYTGYLTNQTLPGRFAQEIGGAVILLEHRYWGTSSPYTNLNTETLQYLTLEQSIADLTHFAKTVDLAFDSNHSSNADKAPWVLTGGSYSGALSAWTASTAPGTFWAYHSSSAPVEAIYNFWQYFVPVVEGMPRNCSMDVSRVVEYVDQVYKSGDKRRQQKLKEMFGLGALQHFDDFAAALENGPWLWQSNSFYTGYSEFYQFCDMVENVQPGAKTVPGPQGVGLEKALKGYASWFKSSFLPGYCAGFGYWTDKLAIDCFDTHKPSNPIFTDQSLANTGNRQWTWLLCNEPLFYWQDGAPPTEITVVSRLVSAEYWQRQCQLYFPEINGHTYGSAEGKRASDVNKWTKGWDSTDTKRLIWTNGQYDPWRDSGVSSVFRPGGPLRSTKQAPVQVIPGGFHCSDLRLRNGQVNAGVQKVIDNEVAQIKAWVKEYPKHAHHHHHH

SEQ ID NO: 2 - protein, 613 aa, Botryotinia fuckeliana
MRFPSIFTAVLFAASSALAAPVNTTTEDETAQIPAEAVIGYSDLEGDFDVAVLPFSNSTNNGLLFINTTIASIAAKEEGVSLEKRAAEHPFLRKGRLIPPVEAEDEFPVSANAAVVNTTGSAFFTQLLDHDNPSKGTFQQKFWWNSEFWAGPGSPIVFFTPGEIAAANYGAYLTNVTVTGLFAQEIKGAVVMVEHRFWGESSPYDNLTTTNLQLLTLKQAIADFVHFAKTVDLPFDSNHSSNAASAPWINSGGSYSGALSAWTESTSPGTFWAYHASSAPVQAIDDYWQYFYPVQDGMPKNCSKDVSLVIDYMDNVLTHGNKSAVTALKTKFGLESVEHNDDFMAVLENGPWLWQSNSFSTGYSGFYQFCDAIENVTAGAAVTPDANGVGLTTALEGYAKWTKSYIPGYCEGFGYDADDLSCLNTHDFNNLMFRDYSVGNAIDRQWNWMLCNEPFGYWQDGAPKNRPTIVSRLVDANYWQRQCALFFPTEGNYTYASAKGATVKRVNKVTKGWDLENTTRLIWTNGQYDPWRTSGVSSQFRPGGELKSTAKHPVQIIPGGFHCSDLRLKNGQVNAGVQKVIDNEVAQIVAWTAEYYNQTSTRPSYGGGHRRRY

SEQ ID NO: 3 - protein, 606 aa, Gibberella zeae
MRFPSIFTAVLFAASSALAAPVNTTTEDETAQIPAEAVIGYSDLEGDFDVAVLPFSNSTNNGLLFINTTIASIAAKEEGVSLEKRAALSLKQEARFETPAKAQLHARQADSTAGVVDGVFHQLVDHDNPSLGTFEQRYWYSLNYANGSNPPVVFISPLDAEAEQVKFWLHDDYVIGGMIARRIGAVMIMLEDRYFGKSSPYDQLTTENMKYYTEDQMVRDKIHFAKTAELPFAKNGGSRPDQVPWVHTGCSAQGNRVMFSQKESPDTFWASWASSAPPQAIPNYWRYFDAAKAYLPKNCTADVEKVIEHLDDVMLNGSADDIQKIKTDFGAPDLKHNDDFMNLLNYGPQTFQGASLRIGDTWQFCDYVENAVDTTDKSKLPGAEGVGLDKALKGYARWTKEVWIPGRCEQQGPWKGENNTGCFNFGDADSLVYATKGLDAPSIVDTLQAQWLFCNEPDENWQTGSPKGTPTLVSRLVNTDYFRKTCARYFPTGPNGETFGLAKGKTADIWNTRYGGWSDPIGYLNRTVLVNGKFDPWRAASFASDQRPGGILGNSTYVKHFINPMGNHCTDTYRNAGSIWPEVKAVQEAGIKQIEKWIAMFPKHKV

SEQ ID NO: 4 - protein, 613 aa, Gibberella zeae
MRFPSIFTAVLFAASSALAAPVNTTTEDETAQIPAEAVIGYSDLEGDFDVAVLPFSNSTNNGLLFINTTIASIAAKEEGVSLEKRPYTVPALSARAKDSGPKAVNISVPVDHFHNETIYEPHSDKKFPLRYWFDAQYYRKGGPVIILASGETSGEDRIPFLEHGILQMLANATGGIGVILEHRYYGTSFPVPDLKPENMRFLSTEQALADTAYFAQHVEFPGMEEHNLTASTTPYIIYGGSYAGAFAAFARKIYPDLFWGGISSSGVTEAIVDYWQYFEAARLFAPGDCAKVTQKLTHAVDNILLGDDKEEKKQLKIAFGLLGLRDDDFAMTISQGIGGLQSNNWDPASDSSSFGLYCGSVSSDDILFASTRPLAPYVKKWLISAGYKKQLKYMTNRFLNYIGYIRSNVESDKSGRCDGKTLDQCYSIRGSMNDTKLDPNNMSRQWTYQTCTQWGYWQTGSGAPKDQLPMVSRLIDVEYNTIPCREEFNITTPPNVESINKLGGFNFSYPRVAFIDGEYDPWRAATPHAIGLPERESTASEPFILIPYGVHHWDENGLAPGSEEIGLPPPAVKQAQQDIIDFTKAWLEDWEKEKGGATADLVPRGSAHHHHHH

SEQ ID NO: 5 - protein, 602 aa, Sclerotinia sclerotiorum
MRFPSIFTAVLFAASSALAAPVNTTTEDETAQIPAEAVIGYSDLEGDFDVAVLPFSNSTNNGLLFINTTIASIAAKEEGVSLEKREHPFLKLRKLVPPVEADDEFPASINAATNITGSAFFTQLLDHENPSKGTFQQKFWWNSENWAGPGSPIVFFTPGEIAAAEYGAYLTNVTVTGLFAQEVKGAVVMVEHRYWGESSPYDNLTTTNLQYLNLKQAIADFVHFAKTVDLPFDTNHSSNAAAAPWILSGGSYSGALAAWTESTSPGTFWAYHASSAPVQAINNYWQYFYPVQDGMAKNCSKDISLVIDYMDNVLTHGNKSAVTALKTKFGLESVTHNDDFMAVLESGPWLWQSNSFTTGYSGFFQFCDAIENVTAGAAVTPDANGVGLTTALEGFAKWTKSLIPGICEDYGYDADDLSCLNTYDFNNFMFRDYSVGNAADRQWNWMLCNEPFGYWQDGAPSNKPTLVSRLINAKYWQRQCALYFPAEGKYTYASAKGATVKQVNQYTQGWNLENTTRLIWTNGQYDPWRTSGVSSQFRPGGELQSTAQHPLQIIPGGFHCSDLRLSNGKANAGVQKVIDNEVAQIVAWTAEYYNKTSSYHSY

SEQ ID NO: 6 - protein, 598 aa, Mycosphaerella graminicola
MRFPSIFTAVLFAASSALAAPVNTTTEDETAQIPAEAVIGYSDLEGDFDVAVLPFSNSTNNGLLFINTTIASIAAKEEGVSLEKRAHRWMHRRAAGDEITPKNIYYSIFDQLIDHNDPSRGTFQQRYWFQFKTWKGPGSPIVLYAPSENNATRDVGFMLPQYGTHGVLAKELGAACVVLEHRFYGNSSPVADLSVENLKDLTLDNVLQDITYFANNVKLPWTNSSSTARDVPWVLMGASYPGSLTSWTANLNPGVFWAYWASSAAMQAIEDFWQYFVPAQQGLPRNCSTDFSQVIDYMDDVVLHGTPQAVTQLKSRFGLEDVSRNDDFMYIFGSVVAALWQGHQFIAPDHSTFPEVFQWCDYIENAVNSSHPLPGAQGVGVTAALDGFVAWWKARGKAYVRQHSSCPEDMSDQMCFDNHNASSPAYTDISVGNPYNRQYFWLDCNTPWGYWQTGAPQGRPSLASRLHTVAYEREQCGMLFPGVEYGQGRNAEDWNAYTGGWKEPPPNSRIMYVNGEFDPWREAGVSSDFRPGGPLQSNEQAKWVVKVVPGGLHTSDMIQDNVRANAGVKQVVDEAVKQMKDWVGEWYEEKGKRPPWEV

SEQ ID NO: 7 - protein, 614 aa, Neurospora crassa
MRFPSIFTAVLFAASSALAAPVNTTTEDETAQIPAEAVIGYSDLEGDFDVAVLPFSNSTNNGLLFINTTIASIAAKEEGVSLEKRAAFFKTLGMEIGPIDDHIESLNKRAEVEGTSGYGTFDQLIDHNTPELGTFKQRFWYGFQYWKGPGSPIILVNPGEQAADGFNKSYLSDQRLAGWMAKDMGAAVVIMEHRYWGNSSPFDELTVKNLQYLTLENSLKDINYFAEHIDLPFDKTNGSKPANAPWIFSGGSYSGALAGWLEALYPGTFWAYHGTSGVVETLGHFWTYFVPVLEATPQNCTKDLTAVIDYVDSVLLHGTPKAKRELKSKFKLQNLTDADFASAIESGPWSWQSTQFYSEKITGYNPYYRFCDYVENVWPNSTNKVPGPLGVGIKKALDGYAKWFVEESLPGTCESSGYDAFKGEDNVLCFQNQNASNPIFHDLSVDNAYNRQWNWFLCNEPFEWWQDGAPLGRPSIVSRLVDADYWRKQCPLWFPAEKGSNATYGIKQGKRAEDVNKWTGGWKHTNGTRIMQANGSLDPWRDVTLSSKFRPGGPFKGNKNHQVRVIEGGTHCSDFYGPNWSANEGIKKVAEEEVAQMGQWVADFYKKRGITRTR

SEQ ID NO: 8 - protein, 592 aa, Talaromyces stipitatus
MRFPSIFTAVLFAASSALAAPVNTTTEDETAQIPAEAVIGYSDLEGDFDVAVLPFSNSTNNGLLFINTTIASIAAKEEGVSLEKRAVRPNIPRPPASAAPAAFEYSTGWFDQLLDHDKPELGTFRQRYFYSTQYWKGSGSPVILFQPGEQTADGFQGYLTNVTISGVYAQEFGGAGIILEHRYWGESSPVNTLTPKTMQHLTFKNALADAVHFAKNVKLPFDNSTRSSPKNAPWILVGGSYSGAQAGWTAATLPGTFWAYHASSAPVEAIWNYWQYFVPIQQRLPKNCSTDLVNVIDHIDSILTGSNESAKDDLKRKFMLGDLRDDDFAAAIVGGPYLGQTTSWGPSGVIYEFCDYIENVHATPPANVSSSGVGVTKALEGYAQWWTTTFFPGTCASYGYWTDQYETACYDTYNSSNPLYADRSINNVADRQWIWFCCNEPFGAWQDGAPDGVPSIVSRLVTADYYLRTCGTYFQPDDGYTYASADGKRPDFLNSWTQGWTGTTERVIWAQGQYDPWREETISSDFRPGGPAISSQVNPIYIMPEASHCYDLLWRNGEGNSGVRDVQKKEVEQMKAWIDEWDVSIHKHVRNT

SEQ ID NO: 9 - protein, 520 aa, Aspergillus clavatus
MGAIPQLPVPPPFEIQESSLLSGNARFRQVRGNTTFDQLIDHDNPELGTFQQRFWWSSEFWKGPGSPVVLFTPGEADAPGYTGYLTNQTLPGRFAQEIGGAVILLEHRYWGTSSPYTNLNTETLQYLTLEQSIADLTHFAKTVDLAFDSNHSSNADKAPWVLTGGSYSGALSAWTASTAPGTFWAYHSSSAPVEAIYNFWQYFVPVVEGMPRNCSMDVSRVVEYVDQVYKSGDKRRQQKLKEMFGLGALQHFDDFAAALENGPWLWQSNSFYTGYSEFYQFCDMVENVQPGAKTVPGPQGVGLEKALKGYASWFKSSFLPGYCAGFGYWTDKLAIDCFDTHKPSNPIFTDQSLANTGNRQWTWLLCNEPLFYWQDGAPPTEITVVSRLVSAEYWQRQCQLYFPEINGHTYGSAEGKRASDVNKWTKGWDSTDTKRLIWTNGQYDPWRDSGVSSVFRPGGPLRSTKQAPVQVIPGGFHCSDLRLRNGQVNAGVQKVIDNEVAQIKAWVKEYPKHAHHHHHH

SEQ ID NO: 10 - protein, 535 aa, Botryolinia fuckeliana
MGEHPFLRKGRLIPPVEAEDEFPVSANAAVVNTTGSAFFTQLLDHDNPSKGTFQQKFWWNSEFWAGPGSPIVFFTPGEIAAANYGAYLTNVTVTGLFAQEIKGAVVMVEHRFWGESSPYDNLTTTNLQLLTLKQAIADFVHFAKTVDLPFDSNHSSNAASAPWINSGGSYSGALSAWTESTSPGTFWAYHASSAPVQAIDDYWQYFYPVQDGMPKNCSKDVSLVIDYMDNVLTHGNKSAVTALKTKFGLESVEHNDDFMAVLENGPWLWQSNSFSTGYSGFYQFCDAIENVTAGAAVTPDANGVGLTTALEGYAKWTKSYIPGYCEGFGYDADDLSCLNTHDFNNLMFRDYSVGNAIDRQWNWMLCNEPFGYWQDGAPKNRPTIVSRLVDANYWQRQCALFFPTEGNYTYASAKGATVKRVNKVTKGWDLENTTRLIWTNGQYDPWRTSGVSSQFRPGGELKSTAKHPVQIIPGGFHCSDLRLKNGQVNAGVQKVIDNEVAQIVAWTAEYYNQTSTRPSYGGGHRRRYAHHHHHH

SEQ ID NO: 11 - protein, 528 aa, Gibberella zeae
MGLSLKQEARFETPAKAQLHARQADSTAGVVDGVFHQLVDHDNPSLGTFEQRYWYSLNYANGSNPPVVFISPLDAEAEQVKFWLHDDYVIGGMIARRIGAVMIMLEDRYFGKSSPYDQLTTENMKYYTEDQMVRDKIHFAKTAELPFAKNGGSRPDQVPWVHTGCSAQGNRVMFSQKESPDTFWASWASSAPPQAIPNYWRYFDAAKAYLPKNCTADVEKVIEHLDDVMLNGSADDIQKIKTDFGAPDLKHNDDFMNLLNYGPQTFQGASLRIGDTWQFCDYVENAVDTTDKSKLPGAEGVGLDKALKGYARWTKEVWIPGRCEQQGPWKGENNTGCFNFGDADSLVYATKGLDAPSIVDTLQAQWLFCNEPDENWQTGSPKGTPTLVSRLVNTDYFRKTCARYFPTGPNGETFGLAKGKTADIWNTRYGGWSDPIGYLNRTVLVNGKFDPWRAASFASDQRPGGILGNSTYVKHFINPMGNHCTDTYRNAGSIWPEVKAVQEAGIKQIEKWIAMFPKHKVAHHHHHH

SEQ ID NO: 12 - protein, 529 aa, Gibberella zeae
MGYTVPALSARAKDSGPKAVNISVPVDHFHNETIYEPHSDKKFPLRYWFDAQYYRKGGPVIILASGETSGEDRIPFLEHGILQMLANATGGIGVILEHRYYGTSFPVPDLKPENMRFLSTEQALADTAYFAQHVEFPGMEEHNLTASTTPYIIYGGSYAGAFAAFARKIYPDLFWGGISSSGVTEAIVDYWQYFEAARLFAPGDCAKVTQKLTHAVDNILLGDDKEEKKQLKIAFGLLGLRDDDFAMTISQGIGGLQSNNWDPASDSSSFGLYCGSVSSDDILFASTRPLAPYVKKWLISAGYKKQLKYMTNRFLNYIGYIRSNVESDKSGRCDGKTLDQCYSIRGSMNDTKLDPNNMSRQWTYQTCTQWGYWQTGSGAPKDQLPMVSRLIDVEYNTIPCREEFNITTPPNVESINKLGGFNFSYPRVAFIDGEYDPWRAATPHAIGLPERESTASEPFILIPYGVHHWDENGLAPGSEEIGLPPPAVKQAQQDIIDFTKAWLEDWEKEKGGATADLVPRGSAHHHHHH

SEQ ID NO: 13 - DNA, 1818 bp, Aspergillus clavatus
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaggcctgccattccacaactgcctgttccacctccattcgaaattcaggaatcttctctgttgtctggtaacgcacgttttcgtcaggtcagaggcaataccactttcgatcaactgattgatcacgacaatccagaattgggcactttccagcagagattttggtggtcttccgaattttggaagggtccaggttctcctgttgtcctgttcaccccaggtgaagcagacgccccaggttacactggttacttgaccaatcagactctgcctggccgttttgcacaagagatcggcggtgccgtgattttgctggaacaccgttactggggtacttcttccccatatactaatctgaataccgagaccctgcaatacttgaccctggaacaatctattgccgatctgactcatttcgccaagactgttgacctggcttttgattccaaccattcttccaatgctgataaagctccttgggtgctgaccggcggttcttactccggtgccctgtctgcatggaccgcttctactgctcctggcactttttgggcataccactcttcttctgcaccagtcgaggctatctataacttttggcaatattttgttccagtcgtcgaaggtatgcctcgtaactgctctatggacgtttcccgtgtagtcgaatatgtagatcaagtctacaagtccggtgataaacgtagacagcaaaaactgaaggagatgttcggtctgggtgccttgcagcattttgacgacttcgctgccgctctggaaaatggtccttggctgtggcaatccaactctttttataccggttactccgaattttaccaattttgtgatatggttgagaacgttcaacctggtgccaagaccgttcctggtccacagggtgtcggtttggagaaggctctgaaaggttacgcttcttggtttaagtcttcctttctgccaggttattgcgctggttttggttactggaccgataaactggcaatcgattgttttgatactcacaagccttccaatcctatttttaccgaccaatctctggcaaacactggtaaccgtcagtggacttggctgttgtgcaatgagcctctgttctactggcaagatggtgctccaccaaccgaaattactgtagtatctcgtctggtttccgcagaatattggcaacgtcaatgtcaactgtacttcccagagattaatggtcatacttacggttctgctgagggcaagcgtgcttccgatgtaaacaagtggaccaaaggttgggattccactgacactaagcgtctgatctggaccaacggccagtacgatccttggcgtgattccggtgtttcttctgtattcagaccaggcggtcctctgcgttctactaaacaagcccctgttcaagttattcctggcggttttcattgttccgacctgagattgcgtaatggtcaagtgaacgccggtgttcagaaggtaattgacaatgaagttgcacaaattaaggcatgggttaaggaatacccaaagcatgcccatcaccatcaccatcactaataa

SEQ ID NO: 14 - DNA, 1845 bp, Botryotinia fuckeliana
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggctgccgaacatccattcctgcgtaaaggccgtttgattccacctgtcgaagctgaagacgaatttcctgtttctgcaaacgcagccgtggttaataccactggttccgcattctttactcagctgctggatcatgataatccatctaaaggtactttccagcaaaaattctggtggaactccgagttttgggctggtcctggctctccaattgtgttttttactccaggtgagattgccgcagcaaattacggtgcctacctgactaacgttaccgttactggcctgtttgcccaagagatcaagggtgcagttgtcatggttgagcatagattttggggtgagtcttccccttatgataacctgactaccactaatttgcaactgttgactctgaaacaagctattgccgatttcgttcatttcgctaaaaccgtagacctgccattcgattccaatcattcctctaacgctgcctctgcaccttggattaactccggtggttcctattctggcgccttgtctgcatggactgaatccacttctcctggtactttttgggcataccacgcttcttctgctccagtccaagccatcgatgattactggcagtacttttatcctgttcaagatggtatgcctaaaaactgctccaaggatgtctccctggtgattgattacatggataatgtcttgacccacggtaataagtctgctgttactgctctgaagactaaatttggtctggagtccgtagaacacaacgatgattttatggccgtcttggaaaatggtccttggctgtggcagtctaattccttctctactggttactctggtttttaccaattttgtgacgccattgaaaatgttactgctggtgccgcagtaactcctgatgctaacggtgtcggcctgaccactgctttggagggttatgctaagtggactaagtcctatatccctggttattgtgaaggcttcggttacgacgccgatgatctgtcttgtctgaacacccacgacttcaataacttgatgttcagagactactctgttggtaacgcaattgatcgtcaatggaactggatgctgtgcaacgagccttttggctactggcaagatggtgcaccaaagaacagaccaaccattgtgtctcgtttggtagatgcaaactattggcagagacagtgcgctctgtttttccctactgaaggcaactatacctatgcatctgccaagggtgctaccgtcaagcgtgtaaataaagtcactaaaggttgggatctggagaatactactcgtttgatttggaccaacggtcaatacgatccttggcgtacctctggcgtctcctcccaatttcgtccaggtggcgaactgaaatccaccgctaagcatccagtccaaattattcctggtggtttccattgttctgacttgagactgaaaaatggccaagttaacgcaggcgtccagaaggttattgacaatgaggttgcccaaattgttgcatggactgccgagtactacaatcaaacttccactcgtccatcctatggtggtggtcaccgtagacgttattaatag

SEQ ID NO: 15 - DNA, 1824 bp, Gibberella zeae
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggctgccctgtccttaaagcaagaagcacgtttcgaaactcctgccaaggcccagctgcatgctcgtcaagccgactctaccgcaggtgttgttgacggtgtgttccatcaactggttgaccatgacaacccatctttgggtactttcgaacaacgttattggtattccctgaattatgctaacggctccaatccaccagttgtgtttatttctcctctggatgcagaggctgaacaagtgaaattttggctgcacgacgactacgtgattggcggtatgattgccagacgtattggtgctgttatgattatgctggaggatcgttacttcggtaagtcctctccatacgatcaactgactactgaaaatatgaaatactatactgaagatcagatggttagagataaaattcattttgccaagaccgccgagttgcctttcgccaaaaatggtggctctagaccagaccaagtcccttgggtgcacactggttgctccgcacaaggcaaccgtgtcatgttttcccaaaaagagtctccagacactttctgggcctcttgggcttcctccgcaccaccacaagctattccaaactactggcgttacttcgatgccgctaaagcctatttgccaaagaattgtaccgccgacgtagagaaggtgattgagcatctggatgatgttatgttgaacggttctgctgatgatatccagaagattaaaactgacttcggtgctccagacctgaagcataatgatgacttcatgaacttgctgaactatggccctcagacttttcaaggtgcttccctgcgtattggcgacacttggcaattttgtgattacgttgaaaatgccgttgatactactgacaagtccaagttgccaggtgctgaaggcgtcggtctggataaagcattgaaaggttatgcaagatggactaaggaagtatggattccaggtcgttgcgagcagcaaggtccttggaaaggtgaaaataacaccggctgttttaactttggtgatgcagattctctggtttacgctactaagggtttggacgcaccatccatcgttgacactctgcaggctcaatggctgttctgtaatgaaccagatgagaactggcaaactggttcccctaaaggtactccaactctggtctctagattggtgaataccgattactttcgtaaaacctgtgcccgttacttcccaactggtcctaacggtgagactttcggtctggcaaagggtaaaactgctgatatctggaataccagatatggtggttggtctgaccctattggttatttgaaccgtaccgttctggtaaacggtaaattcgacccttggagagcagcatcttttgcttctgatcagcgtccaggtggtattctgggtaactccacctatgtcaagcacttcattaatccaatgggtaaccattgcaccgatacctaccgtaatgctggttccatttggcctgaagttaaggcagtacaagaggccggtattaagcagatcgagaaatggattgccatgttccctaagcataaggtttaatag

SEQ ID NO: 16 - DNA, 1845 bp, Gibberella zeae
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaggccttatactgttccagctctgtctgctcgtgcaaaagattctggtccaaaagccgttaacatttccgttcctgtggaccatttccataatgagactatttatgaacctcactctgataaaaagttcccattgcgttattggtttgacgcccaatactacagaaagggtggtcctgttattatcctggcttctggcgaaacctctggtgaagatcgtattccatttctggagcatggtattctgcagatgttggctaacgctactggtggtattggtgttattctggaacatagatattatggcacctctttcccagtacctgatctgaagccagaaaatatgcgtttcctgtccactgagcaagccttggcagacactgcttattttgctcagcatgttgagtttccaggtatggaagagcataatctgactgcatctactactccatacattatttatggtggttcttacgccggtgcttttgctgctttcgcacgtaaaatttacccagatttgttttggggcggtatctcttcttccggtgttactgaagctatcgtagattactggcaatactttgaagccgctcgtctgttcgcaccaggcgactgtgctaaggtaactcagaaattgactcatgctgttgataacatcctgctgggtgacgataaggaagagaagaaacaactgaagattgcttttggtctgttgggcctgagagatgacgactttgcaatgactatttctcaaggtattggtggcctgcaatctaacaattgggaccctgcttccgactcttcttccttcggtctgtactgtggctccgtgtcttccgacgatattctgtttgcttctaccagaccactggccccttatgtgaagaagtggttgatttctgctggctataaaaagcagctgaagtatatgactaatcgtttcctgaattacatcggctatatccgttccaatgttgaatctgataaatccggtagatgcgatggtaaaactttggaccaatgttattctattcgtggttctatgaacgacaccaaactggaccctaacaacatgtctcgtcaatggacttatcaaacctgtactcaatggggttactggcaaaccggctccggtgctccaaaggatcagctgccaatggtatccagactgattgatgtagagtataataccattccatgccgtgaggaatttaacattactactcctcctaacgtcgagtccatcaacaagttgggcggtttcaacttttcttatcctcgtgtagcatttatcgacggtgaatacgatccttggcgtgcagctacccctcatgctattggtctgcctgagagagaatctactgcctccgagccattcattctgattccatacggtgttcaccattgggacgagaatggtctggccccaggctccgaggagatcggtttgccaccacctgctgtcaagcaggcccagcaagacattattgattttactaaggcttggctggaagattgggaaaaagaaaagggtggtgctaccgcagacctggttcctagaggttccgcccatcaccatcaccatcactaataa

SEQ ID NO: 17 - DNA, 1566 bp, Aspergillus clavatus
atgggcgccattccacaactgcctgttccacctccattcgaaattcaggaatcttctctgttgtctggtaacgcacgttttcgtcaggtcagaggcaataccactttcgatcaactgattgatcacgacaatccagaattgggcactttccagcagagattttggtggtcttccgaattttggaagggtccaggttctcctgttgtcctgttcaccccaggtgaagcagacgccccaggttacactggttacttgaccaatcagactctgcctggccgttttgcacaagagatcggcggtgccgtgattttgctggaacaccgttactggggtacttcttccccatatactaatctgaataccgagaccctgcaatacttgaccctggaacaatctattgccgatctgactcatttcgccaagactgttgacctggcttttgattccaaccattcttccaatgctgataaagctccttgggtgctgaccggcggttcttactccggtgccctgtctgcatggaccgcttctactgctcctggcactttttgggcataccactcttcttctgcaccagtcgaggctatctataacttttggcaatattttgttccagtcgtcgaaggtatgcctcgtaactgctctatggacgtttcccgtgtagtcgaatatgtagatcaagtctacaagtccggtgataaacgtagacagcaaaaactgaaggagatgttcggtctgggtgccttgcagcattttgacgacttcgctgccgctctggaaaatggtccttggctgtggcaatccaactctttttataccggttactccgaattttaccaattttgtgatatggttgagaacgttcaacctggtgccaagaccgttcctggtccacagggtgtcggtttggagaaggctctgaaaggttacgcttcttggtttaagtcttcctttctgccaggttattgcgctggttttggttactggaccgataaactggcaatcgattgttttgatactcacaagccttccaatcctatttttaccgaccaatctctggcaaacactggtaaccgtcagtggacttggctgttgtgcaatgagcctctgttctactggcaagatggtgctccaccaaccgaaattactgtagtatctcgtctggtttccgcagaatattggcaacgtcaatgtcaactgtacttcccagagattaatggtcatacttacggttctgctgagggcaagcgtgcttccgatgtaaacaagtggaccaaaggttgggattccactgacactaagcgtctgatctggaccaacggccagtacgatccttggcgtgattccggtgtttcttctgtattcagaccaggcggtcctctgcgttctactaaacaagcccctgttcaagttattcctggcggttttcattgttccgacctgagattgcgtaatggtcaagtgaacgccggtgttcagaaggtaattgacaatgaagttgcacaaattaaggcatgggttaaggaatacccaaagcatgcccatcaccatcaccatcactaataa

SEQ ID NO: 18 - DNA, 1611 bp, Botryotinia fuckeliana
atgggcgaacatccattcctgcgtaaaggccgtttgattccacctgtcgaagctgaagacgaatttcctgtttctgcaaacgcagccgtggttaataccactggttccgcattctttactcagctgctggatcatgataatccatctaaaggtactttccagcaaaaattctggtggaactccgagttttgggctggtcctggctctccaattgtgttttttactccaggtgagattgccgcagcaaattacggtgcctacctgactaacgttaccgttactggcctgtttgcccaagagatcaagggtgcagttgtcatggttgagcatagattttggggtgagtcttccccttatgataacctgactaccactaatttgcaactgttgactctgaaacaagctattgccgatttcgttcatttcgctaaaaccgtagacctgccattcgattccaatcattcctctaacgctgcctctgcaccttggattaactccggtggttcctattctggcgccttgtctgcatggactgaatccacttctcctggtactttttgggcataccacgcttcttctgctccagtccaagccatcgatgattactggcagtacttttatcctgttcaagatggtatgcctaaaaactgctccaaggatgtctccctggtgattgattacatggataatgtcttgacccacggtaataagtctgctgttactgctctgaagactaaatttggtctggagtccgtagaacacaacgatgattttatggccgtcttggaaaatggtccttggctgtggcagtctaattccttctctactggttactctggtttttaccaattttgtgacgccattgaaaatgttactgctggtgccgcagtaactcctgatgctaacggtgtcggcctgaccactgctttggagggttatgctaagtggactaagtcctatatccctggttattgtgaaggcttcggttacgacgccgatgatctgtcttgtctgaacacccacgacttcaataacttgatgttcagagactactctgttggtaacgcaattgatcgtcaatggaactggatgctgtgcaacgagccttttggctactggcaagatggtgcaccaaagaacagaccaaccattgtgtctcgtttggtagatgcaaactattggcagagacagtgcgctctgtttttccctactgaaggcaactatacctatgcatctgccaagggtgctaccgtcaagcgtgtaaataaagtcactaaaggttgggatctggagaatactactcgtttgatttggaccaacggtcaatacgatccttggcgtacctctggcgtctcctcccaatttcgtccaggtggcgaactgaaatccaccgctaagcatccagtccaaattattcctggtggtttccattgttctgacttgagactgaaaaatggccaagttaacgcaggcgtccagaaggttattgacaatgaggttgcccaaattgttgcatggactgccgagtactacaatcaaacttccactcgtccatcctatggtggtggtcaccgtagacgttatgcccatcaccatcaccatcactaataa

SEQ ID NO: 19 - DNA, 1590 bp, Gibberella zeae
atgggcctgtccttaaagcaagaagcacgtttcgaaactcctgccaaggcccagctgcatgctcgtcaagccgactctaccgcaggtgttgttgacggtgtgttccatcaactggttgaccatgacaacccatctttgggtactttcgaacaacgttattggtattccctgaattatgctaacggctccaatccaccagttgtgtttatttctcctctggatgcagaggctgaacaagtgaaattttggctgcacgacgactacgtgattggcggtatgattgccagacgtattggtgctgttatgattatgctggaggatcgttacttcggtaagtcctctccatacgatcaactgactactgaaaatatgaaatactatactgaagatcagatggttagagataaaattcattttgccaagaccgccgagttgcctttcgccaaaaatggtggctctagaccagaccaagtcccttgggtgcacactggttgctccgcacaaggcaaccgtgtcatgttttcccaaaaagagtctccagacactttctgggcctcttgggcttcctccgcaccaccacaagctattccaaactactggcgttacttcgatgccgctaaagcctatttgccaaagaattgtaccgccgacgtagagaaggtgattgagcatctggatgatgttatgttgaacggttctgctgatgatatccagaagattaaaactgacttcggtgctccagacctgaagcataatgatgacttcatgaacttgctgaactatggccctcagacttttcaaggtgcttccctgcgtattggcgacacttggcaattttgtgattacgttgaaaatgccgttgatactactgacaagtccaagttgccaggtgctgaaggcgtcggtctggataaagcattgaaaggttatgcaagatggactaaggaagtatggattccaggtcgttgcgagcagcaaggtccttggaaaggtgaaaataacaccggctgttttaactttggtgatgcagattctctggtttacgctactaagggtttggacgcaccatccatcgttgacactctgcaggctcaatggctgttctgtaatgaaccagatgagaactggcaaactggttcccctaaaggtactccaactctggtctctagattggtgaataccgattactttcgtaaaacctgtgcccgttacttcccaactggtcctaacggtgagactttcggtctggcaaagggtaaaactgctgatatctggaataccagatatggtggttggtctgaccctattggttatttgaaccgtaccgttctggtaaacggtaaattcgacccttggagagcagcatcttttgcttctgatcagcgtccaggtggtattctgggtaactccacctatgtcaagcacttcattaatccaatgggtaaccattgcaccgatacctaccgtaatgctggttccatttggcctgaagttaaggcagtacaagaggccggtattaagcagatcgagaaatggattgccatgttccctaagcataaggttgcccatcaccatcaccatcactaataa

SEQ ID NO: 20 - DNA, 1593 bp, Gibberella zeae
atgggctatactgttccagctctgtctgctcgtgcaaaagattctggtccaaaagccgttaacatttccgttcctgtggaccatttccataatgagactatttatgaacctcactctgataaaaagttcccattgcgttattggtttgacgcccaatactacagaaagggtggtcctgttattatcctggcttctggcgaaacctctggtgaagatcgtattccatttctggagcatggtattctgcagatgttggctaacgctactggtggtattggtgttattctggaacatagatattatggcacctctttcccagtacctgatctgaagccagaaaatatgcgtttcctgtccactgagcaagccttggcagacactgcttattttgctcagcatgttgagtttccaggtatggaagagcataatctgactgcatctactactccatacattatttatggtggttcttacgccggtgcttttgctgctttcgcacgtaaaatttacccagatttgttttggggcggtatctcttcttccggtgttactgaagctatcgtagattactggcaatactttgaagccgctcgtctgttcgcaccaggcgactgtgctaaggtaactcagaaattgactcatgctgttgataacatcctgctgggtgacgataaggaagagaagaaacaactgaagattgcttttggtctgttgggcctgagagatgacgactttgcaatgactatttctcaaggtattggtggcctgcaatctaacaattgggaccctgcttccgactcttcttccttcggtctgtactgtggctccgtgtcttccgacgatattctgtttgcttctaccagaccactggccccttatgtgaagaagtggttgatttctgctggctataaaaagcagctgaagtatatgactaatcgtttcctgaattacatcggctatatccgttccaatgttgaatctgataaatccggtagatgcgatggtaaaactttggaccaatgttattctattcgtggttctatgaacgacaccaaactggaccctaacaacatgtctcgtcaatggacttatcaaacctgtactcaatggggttactggcaaaccggctccggtgctccaaaggatcagctgccaatggtatccagactgattgatgtagagtataataccattccatgccgtgaggaatttaacattactactcctcctaacgtcgagtccatcaacaagttgggcggtttcaacttttcttatcctcgtgtagcatttatcgacggtgaatacgatccttggcgtgcagctacccctcatgctattggtctgcctgagagagaatctactgcctccgagccattcattctgattccatacggtgttcaccattgggacgagaatggtctggccccaggctccgaggagatcggtttgccaccacctgctgtcaagcaggcccagcaagacattattgattttactaaggcttggctggaagattgggaaaaagaaaagggtggtgctaccgcagacctggttcctagaggttccgcccatcaccatcaccatcactaataa

SEQ ID NO: 21 - DNA, 1812 bp, Sclerotinia sclerotiorum
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggaacacccattcttgaagttgagaaagttggtccctccagttgaagccgatgatgagttcccagcctccattaacgcagccactaacattactggttctgctttctttacacaattgcttgatcatgaaaatccatccaagggaacttttcaacagaaattctggtggaactcagagaattgggcaggtccaggaagtcctattgttttctttacacctggtgaaatcgctgccgcagagtacggagcctatttgaccaacgttactgtcacaggtctttttgctcaagaagttaagggagccgttgtcatggtcgaacacagatactggggagagtcttccccatacgataacttgactacaaccaatcttcaatacttgaaccttaagcaggccattgcagattttgttcatttcgctaaaacagtcgatttgccattcgacaccaaccactcaagtaatgctgccgcagctccttggatcttgtctggtggatcatatagtggtgctttggccgcatggaccgagtctacttccccaggaactttttgggcctaccatgcatcttccgctcctgttcaagctattaacaactactggcaatacttctatccagttcaggacggtatggccaagaattgttctaaggatatttccttggttatcgattacatggacaatgtcttgactcatggtaacaaatcagccgttacagcattgaagaccaaatttggacttgaaagtgttacacacaatgatgacttcatggctgtcttggagtccggtccttggctttggcaatcaaacagttttactacaggttattccggatttttccagttctgcgacgcaattgaaaatgttaccgctggtgctgccgtcactccagatgctaacggtgttggattgaccactgcacttgaaggatttgctaagtggactaaatctttgattcctggtatctgtgaggattacggatatgacgctgatgacttgtcctgccttaacacttacgatttcaacaacttcatgttcagagactattcagttggtaacgcagctgatagacaatggaattggatgttgtgtaacgagccattcggttactggcaagatggagccccatctaataagcctactttggtttccagacttattaacgcaaaatactggcaaagacagtgcgccttgtattttcctgcagagggtaaatacacctatgcttctgccaagggagctactgttaaacaagtcaatcagtacacacaaggttggaacttggagaatacaaccagacttatctggaccaacggtcaatatgatccttggagaacttcaggagtttcaagtcagtttagacctggtggagaattgcagtctactgctcaacatccattgcagattatccctggtggattccactgttcagacttgagacttagtaacggtaaagcaaatgctggagttcaaaaagtcattgataatgaagttgctcagatcgtcgcttggactgccgagtactataacaaaacatcttcctaccactcttattaataa

SEQ ID NO: 22 - DNA, 1800 bp, Mycosphaerella graminicola
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggcacacagatggatgcacagaagagccgcaggtgacgaaattacccctaaaaatatctattacagtatctttgaccagttgattgaccataacgatccatccagaggtacttttcaacagagatactggtttcaattcaagacctggaaaggtccaggttctccaattgttttgtatgccccttcagaaaacaatgcaactagagacgttggttttatgcttccacaatacggtacacatggagtcttggccaaggaacttggtgctgcctgtgttgtcttggagcacagattctatggaaactcttcccctgttgctgatttgtccgtcgagaaccttaaggatttgacccttgacaatgttttgcaagacattacttacttcgctaacaacgtcaaacttccttggaccaattcaagttctactgcaagagatgttccttgggtcttgatgggtgcttcttatccaggaagtttgacttcttggacagctaaccttaatcctggtgttttctgggcctactgggcatcctcagcagctatgcaagctattgaagacttttggcagtatttcgttcctgcccaacagggattgccaagaaactgtagtactgatttctctcaagttatcgactacatggatgacgttgtcttgcatggtacaccacaagctgttacccagttgaaaagtagattcggacttgaggatgtttccagaaacgatgacttcatgtacatcttcggttccgttgtcgccgcattgtggcaaggacatcagtttatcgctcctgatcactcaacctttccagaagttttccaatggtgcgactatattgagaacgccgtcaatagttctcaccctttgccaggtgcacagggtgttggagtcactgctgcccttgatggtttcgttgcttggtggaaggccagaggaaaagcatacgtcagacaacattcctcatgtccagaagatatgtcagaccagatgtgctttgataaccacaatgctagttctcctgcctatactgacatctctgttggtaacccatacaatagacaatatttctggttggattgtaacactccttggggatactggcaaacaggtgctcctcagggaagaccatccttggcttcaagacttcatacagttgcctatgaaagagagcaatgcggaatgttgtttccaggagttgaatacggtcagggaagaaacgcagaggattggaatgcttatactggtggatggaaggaaccacctccaaactccagaatcatgtacgttaacggtgaatttgatccttggagagaggcaggagtttcctcagacttcagacctggtggaccattgcaaagtaatgagcaggctaagtgggttgtcaaagttgtcccaggtggacttcacacttccgatatgatccaagacaacgttagagcaaatgctggtgtcaaacaagttgtcgatgaagctgttaagcagatgaaagactgggtcggagagtggtatgaagagaagggaaaaagacctccttgggaagtttaataa

SEQ ID NO: 23 - DNA, 1848 bp, Neurospora crassa
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggcagcattctttaagacattgggtatggaaattggtcctattgacgatcacattgagagtttgaacaagagagccgaagttgagggaacttcaggttacggaacatttgatcaattgattgaccataacaccccagagcttggaacttttaaacaaagattctggtacggttttcagtattggaagggtccaggatctcctattatcttggttaaccctggtgaacaagctgccgatggttttaacaagtcttacttgtccgaccagagacttgccggatggatggcaaaggatatgggtgcagctgttgtcattatggagcatagatattggggtaactcttccccattcgatgaattgacagttaaaaatcttcaatacttgacccttgagaactctcttaaggacattaactacttcgctgaacacatcgatttgccttttgacaaaactaacggttctaagccagccaatgcaccttggatcttctccggtggatcatacagtggtgctttggccggatggttggaagctctttacccaggaactttttgggcctatcatggtacatctggagttgtcgagaccttgggtcacttctggacttactttgttccagtccttgaagcaactcctcaaaactgtacaaaagatttgaccgctgttattgattatgttgactcagtcttgcttcacggtacaccaaaggcaaagagagagttgaagagtaaattcaagttgcaaaatcttaccgatgccgactttgcatcagctattgagagtggaccttggtcatggcaaagtactcagttctactctgaaaagatcacaggttacaacccatactacagattctgcgattacgttgaaaatgtctggcctaactccactaacaaggttccaggtcctttgggtgtcggaattaagaaagctcttgacggttacgccaagtggttcgttgaagagtctttgcctggtacttgtgagtcaagtggatatgatgctttcaaaggtgaagacaacgttttgtgctttcaaaaccagaatgcctctaatccaatttttcatgatttgtccgtcgacaacgcatataatagacaatggaactggttcttgtgtaatgagccttttgaatggtggcaggatggtgctccattgggaagaccttctatcgtttccagacttgtcgatgccgactactggagaaaacaatgcccattgtggtttcctgccgagaagggttccaacgcaacctatggtattaaacagggaaagagagctgaagatgttaacaagtggactggtggatggaagcacaccaacggaactagaatcatgcaagctaatggttctttggacccttggagagacgttactctttcttccaagttcagaccaggtggaccttttaaaggaaacaagaatcatcaggttagagtcattgagggtggaacacactgttcagatttctacggacctaactggagtgctaatgaaggtatcaagaaagttgcagaagaggaagtcgctcaaatgggacagtgggttgctgacttctacaagaaaagaggtatcactagaacaagataataa

SEQ ID NO: 24 - DNA, 1782 bp, Talaromyces stipitatus
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagggccgtcagacctaacatccctagacctcctgcttccgccgctccagccgcatttgaatactccaccggttggtttgatcagttgcttgaccatgataagccagaattgggtacttttagacaaagatacttctactctacccagtactggaaaggtagtggatctccagttatcttgtttcaacctggagaacagactgctgatggtttccaaggataccttactaacgtcacaatttctggtgtttatgctcaggagtttggtggagccggaattatcttggaacacagatactggggagagtcttccccagttaacacacttacccctaagaccatgcaacatttgactttcaaaaatgcacttgctgacgccgtccactttgctaagaacgttaaattgcctttcgataactctactagatcaagtccaaagaatgctccttggattcttgttggtggttcttactcaggtgcacaagctggatggaccgctgccactttgccaggtacattttgggcttatcatgcctcttccgcacctgtcgaagctatctggaactactggcaatatttcgttccaattcaacagagattgcctaagaactgtagtaccgaccttgttaatgtcattgaccacatcgattccattttgactggaagtaatgagtctgctaaggatgaccttaagagaaagtttatgttgggagatttgagagatgacgatttcgcagctgccattgtcggtggaccatacttgggacaaactacaagttggggtccttctggagttatctacgaattttgcgattacatcgagaacgtccatgctactccacctgccaatgtttcaagttctggtgttggagtcacaaaggcattggaaggttatgctcaatggtggaccactacatttttcccaggtacttgtgcctcctacggatattggacagaccagtacgagaccgcatgctacgatacttataactcctcaaatccattgtatgcagacagatctatcaacaatgttgctgatagacaatggatttggttttgttgcaacgaacctttcggtgcctggcaggacggtgcaccagatggagttccttccattgtctcaagattggttacagctgattactatcttagaacatgtggtacttactttcaaccagacgatggatacacctatgcctccgcagacggtaaaagacctgatttcttgaactcatggactcagggttggacaggaaccactgagagagttatttgggctcaaggacagtatgacccttggagagaagagactatcagttctgattttagaccaggtggacctgctatttcctcacaagttaatccaatctacatcatgcctgaagcctcacactgttatgacttgctttggagaaacggagagggaaattctggtgttagagatgtccaaaagaaagaagttgagcagatgaaggcttggatcgacgaatgggatgtctctatccataagcacgttagaaacacttaataa

SEQ ID NO: 25 - protein, 526 aa, Botryotinia fuckeliana
EHPFLRKGRLIPPVEAEDEFPVSANAAVVNTTGSAFFTQLLDHDNPSKGTFQQKFWWNSEFWAGPGSPIVFFTPGEIAAANYGAYLTNVTVTGLFAQEIKGAVVMVEHRFWGESSPYDNLTTTNLQLLTLKQAIADFVHFAKTVDLPFDSNHSSNAASAPWINSGGSYSGALSAWTESTSPGTFWAYHASSAPVQAIDDYWQYFYPVQDGMPKNCSKDVSLVIDYMDNVLTHGNKSAVTALKTKFGLESVEHNDDFMAVLENGPWLWQSNSFSTGYSGFYQFCDAIENVTAGAAVTPDANGVGLTTALEGYAKWTKSYIPGYCEGFGYDADDLSCLNTHDFNNLMFRDYSVGNAIDRQWNWMLCNEPFGYWQDGAPKNRPTIVSRLVDANYWQRQCALFFPTEGNYTYASAKGATVKRVNKVTKGWDLENTTRLIWTNGQYDPWRTSGVSSQFRPGGELKSTAKHPVQIIPGGFHCSDLRLKNGQVNAGVQKVIDNEVAQIVAWTAEYYNQTSTRPSYGGGHRRRY

SEQ ID NO: 26 - protein, 517 aa, Sclerotinia sclerotiorum
EHPFLKLRKLVPPVEADDEFPASINAATNITGSAFFTQLLDHENPSKGTFQQKFWWNSENWAGPGSPIVFFTPGEIAAAEYGAYLTNVTVTGLFAQEVKGAVVMVEHRYWGESSPYDNLTTTNLQYLNLKQAIADFVHFAKTVDLPFDTNHSSNAAAAPWILSGGSYSGALAAWTESTSPGTFWAYHASSAPVQAINNYWQYFYPVQDGMAKNCSKDISLVIDYMDNVLTHGNKSAVTALKTKFGLESVTHNDDFMAVLESGPWLWQSNSFTTGYSGFFQFCDAIENVTAGAAVTPDANGVGLTTALEGFAKWTKSLIPGICEDYGYDADDLSCLNTYDFNNFMFRDYSVGNAADRQWNWMLCNEPFGYWQDGAPSNKPTLVSRLINAKYWQRQCALYFPAEGKYTYASAKGATVKQVNQYTQGWNLENTTRLIWTNGQYDPWRTSGVSSQFRPGGELQSTAQHPLQIIPGGFHCSDLRLSNGKANAGVQKVIDNEVAQIVAWTAEYYNKTSSYHSY

SEQ ID NO: 27 - protein, 477 aa, Artificial Sequence
  Consensus of the five best polypeptide sequences.
FDQLLDHDNPSLGTFQQKFWWNSEFWAGPGSPIVFFTPGEIAAAGYGAYLTNVTVTGLFAQEIKGAVVMVEHRYWGESSPYDNLTTTNLQYLTLKQAIADFVHFAKTVDLPFDSNHSSNAANAPWILSGGSYSGALSAWTASTSPGTFWAYHASSAPVQAIYNYWQYFYPVQDGMPKNCSKDVSLVIDYMDNVLTHGNKSAVTALKTKFGLESLEHNDDFMAALENGPWLWQSNSFSTGYSGFYQFCDAIENVTAGAAVTPDANGVGLTTALEGYAKWTKSTFIPGYCEGFGYDADDLALSCLNTHDFNNPMFRDYSVGNAADRQWNWMLCNEPFGYWQDGAPDNVPTIVSRLVSAEYWQRQCALYFPAEGGYTYASAKGATVKQVNKWTKGWDLENTTRLIWTNGQYDPWRTSGVSSQFRPGGELKSTAGHPVQIIPGGFHCSDLRLRNGQVNAGVQKVIDNEVAQIVAWTAEYYN

SEQ ID NO: 28 - protein, 511 aa, Aspergillus clavatus
AIPQLPVPPPFEIQESSLLSGNARFRQVRGNTTFDQLIDHDNPELGTFQQRFWWSSEFWKGPGSPVVLFTPGEADAPGYTGYLTNQTLPGRFAQEIGGAVILLEHRYWGTSSPYTNLNTETLQYLTLEQSIADLTHFAKTVDLAFDSNHSSNADKAPWVLTGGSYSGALSAWTASTAPGTFWAYHSSSAPVEAIYNFWQYFVPVVEGMPRNCSMDVSRVVEYVDQVYKSGDKRRQQKLKEMFGLGALQHFDDFAAALENGPWLWQSNSFYTGYSEFYQFCDMVENVQPGAKTVPGPQGVGLEKALKGYASWFKSSFLPGYCAGFGYWTDKLAIDCFDTHKPSNPIFTDQSLANTGNRQWTWLLCNEPLFYWQDGAPPTEITVVSRLVSAEYWQRQCQLYFPEINGHTYGSAEGKRASDVNKWTKGWDSTDTKRLIWTNGQYDPWRDSGVSSVFRPGGPLRSTKQAPVQVIPGGFHCSDLRLRNGQVNAGVQKVIDNEVAQIKAWVKEYPKH

SEQ ID NO: 29 - protein, 506 aa, Talaromyces stipitatus
VRPNIPRPPASAAPAAFEYSTGWFDQLLDHDKPELGTFRQRYFYSTQYWKGSGSPVILFQPGEQTADGFQGYLTNVTISGVYAQEFGGAGIILEHRYWGESSPVNTLTPKTMQHLTFKNALADAVHFAKNVKLPFDNSTRSSPKNAPWILVGGSYSGAQAGWTAATLPGTFWAYHASSAPVEAIWNYWQYFVPIQQRLPKNCSTDLVNVIDHIDSILTGSNESAKDDLKRKFMLGDLRDDDFAAAIVGGPYLGQTTSWGPSGVIYEFCDYIENVHATPPANVSSSGVGVTKALEGYAQWWTTTFFPGTCASYGYWTDQYETACYDTYNSSNPLYADRSINNVADRQWIWFCCNEPFGAWQDGAPDGVPSIVSRLVTADYYLRTCGTYFQPDDGYTYASADGKRPDFLNSWTQGWTGTTERVIWAQGQYDPWREETISSDFRPGGPAISSQVNPIYIMPEASHCYDLLWRNGEGNSGVRDVQKKEVEQMKAWIDEWDVSIHKHVRNT

SEQ ID NO: 30 - protein, 528 aa, Neurospora crassa
AFFKTLGMEIGPIDDHIESLNKRAEVEGTSGYGTFDQLIDHNTPELGTFKQRFWYGFQYWKGPGSPIILVNPGEQAADGFNKSYLSDQRLAGWMAKDMGAAVVIMEHRYWGNSSPFDELTVKNLQYLTLENSLKDINYFAEHIDLPFDKTNGSKPANAPWIFSGGSYSGALAGWLEALYPGTFWAYHGTSGVVETLGHFWTYFVPVLEATPQNCTKDLTAVIDYVDSVLLHGTPKAKRELKSKFKLQNLTDADFASAIESGPWSWQSTQFYSEKITGYNPYYRFCDYVENVWPNSTNKVPGPLGVGIKKALDGYAKWFVEESLPGTCESSGYDAFKGEDNVLCFQNQNASNPIFHDLSVDNAYNRQWNWFLCNEPFEWWQDGAPLGRPSIVSRLVDADYWRKQCPLWFPAEKGSNATYGIKQGKRAEDVNKWTGGWKHTNGTRIMQANGSLDPWRDVTLSSKFRPGGPFKGNKNHQVRVIEGGTHCSDFYGPNWSANEGIKKVAEEEVAQMGQWVADFYKKRGITRTR

(19) United States
(12) Patent Application Publication
(10) Pub. No.: US 2014/0205587 A1
(43) Pub. Date: Jul. 24, 2014

(54) Proteases for Degrading Gluten

(71) Applicant: Matthew John Siegel, Menlo Park, CA (US)

(72) Inventor: Matthew John Siegel, Menlo Park, CA (US)

(73) Assignee: ALVINE PHARMACEUTICALS, INC., San Carlos, CA (US)

(21) Appl. No.: 14/126,621

(22) PCT Filed: Jun. 13, 2012
(86) PCT No.: PCT/US12/42273

§ 371 Date: Mar. 28, 2014

Related U.S. Application Data
(60) Provisional application No. 61/498,445, filed on Jun. 17, 2011.

(51) Int. Cl. C12N 9/62; A23L 1/29; A23L 1/30; C12N 9/58; A61K 38/48

(52) U.S. Cl. CPC C12N 9/62; C12N 9/58; A61K 38/482; A61K 38/4873; A23L 1/3014; A23L 1/296
USPC 424/94.64; 435/225; 435/223; 435/320.1; 426/18; 426/63

(57) ABSTRACT

Gluten-degrading proteases can be used to degrade gluten and for making gluten-containing food safer for patients suffering from gluten intolerance.

### BACKGROUND OF THE INVENTION

1. Field of the Invention

The present invention provides isolated, purified, and recombinant forms of gluten-degrading proteases and methods for their use in degrading gluten in food. The invention therefore relates to the fields of biology, food preparation, medicine, and molecular biology.

2. Description of Related Disclosures

Celiac disease, also known as celiac sprue, and dermatitis herpetiformis (“DH”) are autoimmune diseases (and may be different manifestations of the same disease), and gluten sensitivity is a condition (collectively, celiac disease, DH, and gluten sensitivity are referred to herein as “gluten intolerance”) triggered by dietary gluten, a storage protein found in wheat and other cereals. Patients diagnosed with gluten intolerance are advised or choose on their own to refrain from consuming gluten in any amount. Because gluten is a common protein in food, however, patients find it very difficult to avoid gluten and frequently experience relapse due to inadvertent exposure.

U.S. Pat. No. 7,303,871 describes therapies for gluten intolerance that involve pre-treatment of gluten-containing food with a protease as well as the use of orally administered proteases to degrade gluten contemporaneously with its ingestion. U.S. Pat. No. 7,320,788 describes admixtures of proteases useful in these therapies, including an admixture of a prolyl endopeptidase (PEP), such as Sphingomonas capsulata PEP, and a glutamine endoprotease, such as EPB2 from barley. One such admixture formulated for oral administration and composed of recombinant forms of the barley EPB2 and the S. capsulata PEP (termed, respectively, ALV001 and ALV002; see PCT Pub. Nos. 2008/1115411 and 2008/115428) is currently in clinical trials. U.S. Pat. Nos. 7,323,327 and 7,309,595 describe certain proteolytic enzymes. Each of the aforementioned patents and patent publications is specifically incorporated herein by reference.

To be effective upon oral administration, a protease must be active or, if in a zymogen form, activate and remain active long enough to degrade any gluten present into non-immunogenic fragments. The immunogenic peptides can be relatively small (˜10 amino acids) and are contained, often in multiple copies, in very large proteins. The conditions in the gastrointestinal tract are harsh, and any exogenously added protease is typically degraded, and so rendered inactive, quickly. Accordingly, there remains a need in the art for proteases useful in the treatment of gluten intolerance. The present invention meets that need.

### SUMMARY OF THE INVENTION

In a first aspect, the present invention provides gluten-degrading, proline-specific proteases, termed “glutenases”, from Botryotinia fuckeliana, Aspergillus clavatus, Sclerotinia sclerotiorum, Mycosphaerella graminicola, Neurospora crassa, Talaromyces stipitatus and Gibberella zeae (abbreviated BF PEP, AC PEP, SS PEP, MG PEP, NC PEP, TS PEP, and GZ PEP, respectively) in isolated, purified, and recombinant form as well as combinations of them with one or more other glutenases. These proteases are homologous to lysosomal Pro-Xaa carboxypeptidases. Proteases of the invention are also provided, in some embodiments, in PEGylated form; see PCT Pub. No. 2007/047303, incorporated herein by reference.

In a second aspect, the present invention provides recombinant expression vectors encoding the proteases of the invention and methods for using such vectors to produce the encoded proteases.

In a third aspect, the present invention provides methods for degrading gluten in food, comprising contacting gluten-containing food with a protease of the invention in an isolated, purified, or recombinant form. Such methods also include the use of the proteases in combinations with other gluten-degrading proteases, e.g. Hordeum vulgare endopeptidase B (EPB2), aspergillopepsin from Aspergillus niger, Hordeum vulgarum endopeptidase C, Sphingomonas capsulata prolyl endopeptidase, and the like. A “combination”, as used herein, refers to two or more proteases (including two or more endopeptidases, a type of protease) that can be administered contemporaneously in separate formulations, or can be co-formulated in accordance with the invention. In some embodiments the protease or combination of proteases is ingested by an individual contemporaneously with food, e.g. at meal time.

In a fourth aspect, the present invention provides pharmaceutical formulations and unit dose forms suitable for oral administration and containing a protease or combination of proteases as provided by the invention, in an isolated, purified, or recombinant form admixed with one or more pharmaceutically acceptable excipients. Suitable excipients include those disclosed in PCT Publication Nos. 2007/044906; 2008/115411; 2010/021752; and 2010/042203, each of which is incorporated herein by reference.

In a fifth aspect, the present invention provides a method for treating gluten intolerance in a patient in need of such treatment, wherein said treatment reduces the exposure of said patient to immunogenic gluten peptides, said method comprising the step of orally administering to said patient a therapeutically effective dose of a protease of the invention in an isolated, purified, or recombinant form, or a combination of proteases that comprises at least one protease of the invention, or a pharmaceutical formulation thereof contemporaneously with the ingestion of a food that may contain gluten. In one embodiment, the patient has celiac disease. In other embodiment, the patient has dermatitis herpetiformis. In another embodiment, the patient has not been diagnosed as having gluten intolerance but simply prefers not to consume gluten or has gluten sensitivity.

These and other aspects and embodiments of the invention are described in more detail below.

### BRIEF DESCRIPTION OF THE DRAWINGS

FIG. 1 provides an alignment and consensus of protease amino acid sequences.

FIGS. 2A-2B provide graphs of pepsin stability of selected prolyl endopeptidase enzymes.

FIGS. 3A-3D provide graphs of pH stability of selected prolyl endopeptidase enzymes.

### DETAILED DESCRIPTION OF THE EMBODIMENTS

The present invention provides gluten-degrading proteases in isolated, purified, and/or recombinant form and combinations comprising one or more of them, including combinations with another gluten-degrading protease, such as EPB2, to reduce the concentration of immunogenic gluten peptides in gluten containing foods. Some of the favorable properties of these proteases with respect to degrading gluten in the gastrointestinal tract include: resistance to degradation by proteases in the gastrointestinal (GI) tract providing longer duration of activity in the GI tract; synergy with other proteases in gluten-degrading activity; broad pH stability and activity range that facilitates optimal activity under acidic gastric conditions; and favorable kinetics enabling rapid degradation of gluten.

In some embodiments of the invention, a glutenase of the invention is derived from Botryotinia fuckeliana, Aspergillus clavatus, Sclerotinia sclerotiorum, Mycosphaerella graminicola, Neurospora crassa, Talaromyces stipitatus or Giberrella zeae. In some embodiments of the methods of the invention one or more of these proteases is used in combination, including in combination with at least one other glutenase.

The amino acid sequences of exemplary proteases are listed by reference to SEQ ID NO and other identifying information in Table 1, and in the sequence listing as proteins (SEQ ID NO:1-12) and encoding nucleotide sequences (SEQ ID NO:13-24). The sequence listing provides the protease amino acid sequence, and in addition, SEQ ID NO:1, 4, 9-12 contain a sequence composed of six histidines (6×his tag) and SEQ ID NO:4, 12 contain a thrombin cleavage site (LVPRGS) is shown at the C-termini of these protease to illustrate one example of a form of the recombinant proteases of the invention. This optional additional sequence facilitates purification using metal affinity chromatography of the recombinant protease containing them. The nucleotide sequences have been modified from the native sequence to be optimized for expression in Pichia pastoris (SEQ ID NO:13-16, 21-24) and Escherichia coli (SEQ ID NO: 17-20).

For expression in Pichia pastoris, the genes of interest were cloned into expression plasmid pPINKalphaHC, which contains the Saccharomyces cerevisiae alpha mating factor for protein secretion into the culture medium. The first 85 amino acids in SEQ ID NO: 1-8 and the first 255 nucleotides in SEQ ID NO:13-16, 21-24 encode the Saccharomyces cerevisiae alpha mating factor and contain a KEX2 cleavage site for post-translational cleavage of this sequence from the remainder of the protein. Regions of the sequences contain restriction sites introduced by recombinant DNA technology (NcoI on 5′ and BamHI on 3′ end) to facilitate cloning into an E. coli expression vector in SEQ ID NO 17-20.

TABLE 1
Proteases of the Invention.

| SEQ ID NO | Pubmed Protein ID/Gene ID | Similarity to |
|---|---|---|
| 1, 9, 13, 17 | XP_001273182/LOC4704745 | Serine Peptidase, Putative |
| 2, 10, 14, 18 | XP_001551952/LOC5432485 | Hypothetical Protein |
| 3, 11, 15, 19 | XP_384993/LOC2786991 | Hypothetical Protein |
| 4, 12, 16, 20 | XP_382380/LOC2781756 | Hypothetical Protein |
| 5, 21 | XP_001595272/LOC5491761 | Hypothetical Protein |
| 6, 22 | EGP83270 | Serine Carboxypeptidase |
| 7, 23 | XP_958301/LOC3874448 | Hypothetical Protein |
| 8, 24 | XP_002484534/LOC8110018 | Serine Peptidase, Putative |

As used herein, a glutenase of the invention is a protease shown in Table 1 or a protease that has homology to a protease shown in Table 1, or a variant of either. Homologous and variant proteases can be naturally occurring or constructed. Thus, the invention provides, in addition to the specific sequences set forth in Table 1, variants and homologs of those sequences. A variant can be substantially similar to a native sequence, i.e. differing by at least one amino acid, or at least two amino acids, or at least ten amino acids, but usually not more than about fifty amino acids (the number of differences depending on the size of the reference sequence). The sequence variations can be substitutions, insertions, or deletions. Scanning mutations that systematically introduce alanine, or other residues, may be used to determine key amino acids to be maintained in variant sequences. Once key amino acids are identified, other amino acids can be changed (typically by making a change in the DNA encoding the protease) in accordance with the invention. For example, one can change a non-key amino acid by making a conservative amino acid substitution. Conservative amino acid substitutions that can be used to generate a variant sequence of the invention typically include substitutions within the following groups: (glycine, alanine); (valine, isoleucine, leucine); (aspartic acid, glutamic acid); (asparagine, glutamine); (serine, threonine); (lysine, arginine); and (phenylalanine, tyrosine).

Homologs of the sequences of the proteases shown in Table 1 typically have at least about 70% sequence identity at the amino acid sequence level, and include proteases that have at least about 80% sequence identity, at least about 90% sequence identity, at least about 95% sequence identity, and at least about 99% sequence identity. Homologs of the proteases in Table 1 include the counterpart proteases in any one of the genera and species from which the proteases in Table 1 are naturally expressed. In various embodiments, a protease of the invention is any protease other than a prolyl endopeptidase from Aspergillus niger, that has at least 50% identity at the amino acid sequence level to a protease in Table 1. In some embodiments, the identity is 70% or higher, as noted above.

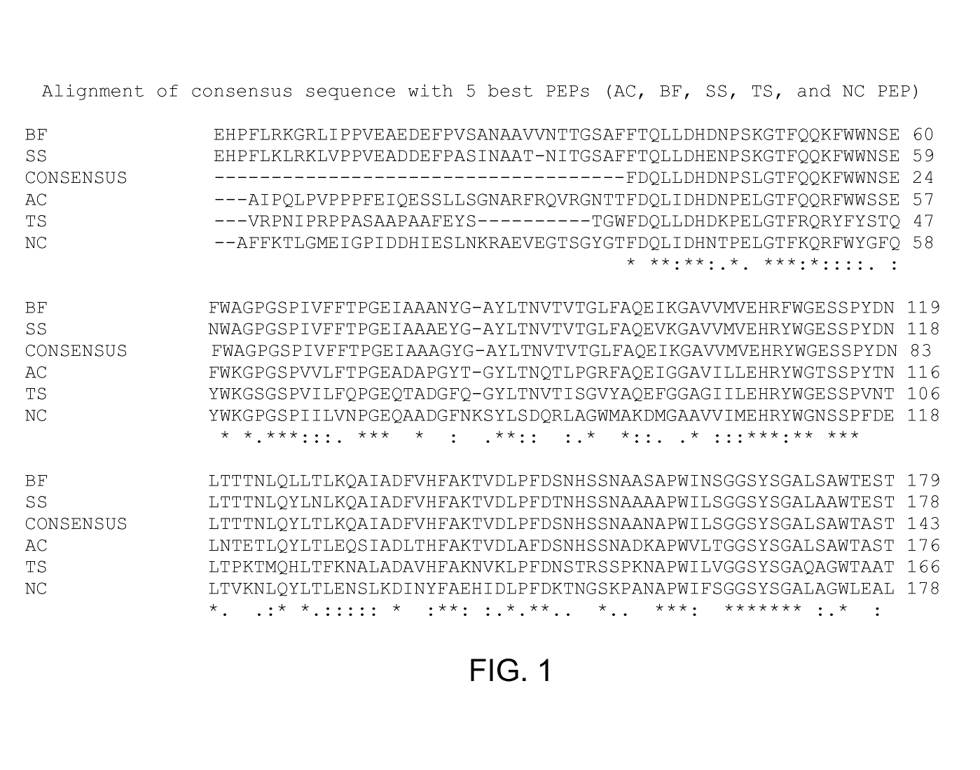

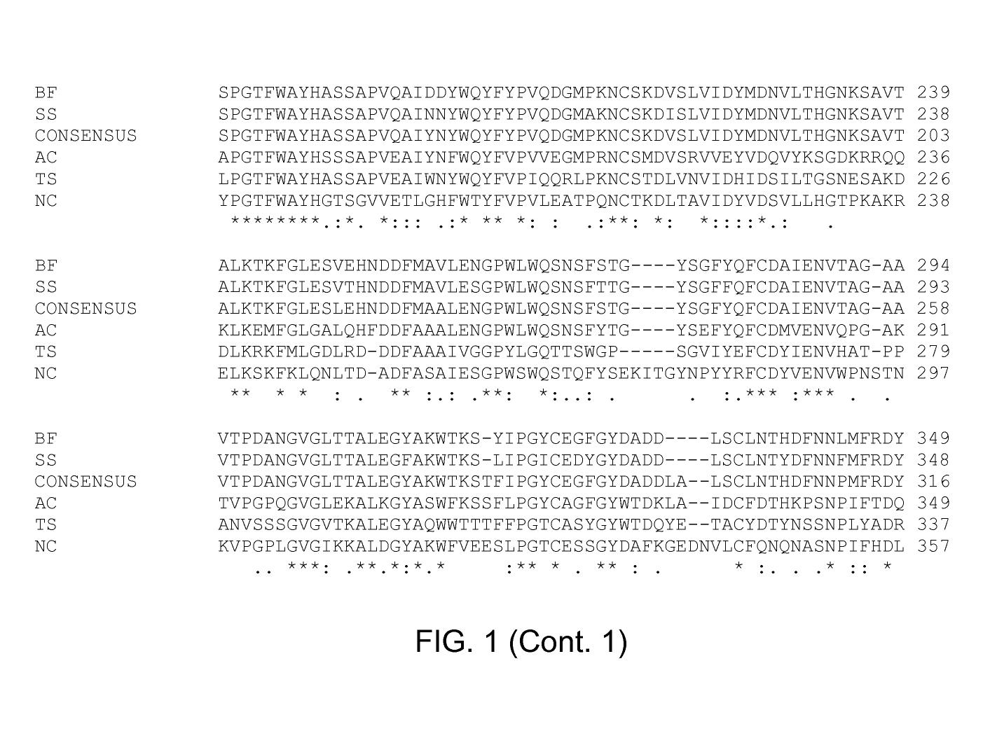

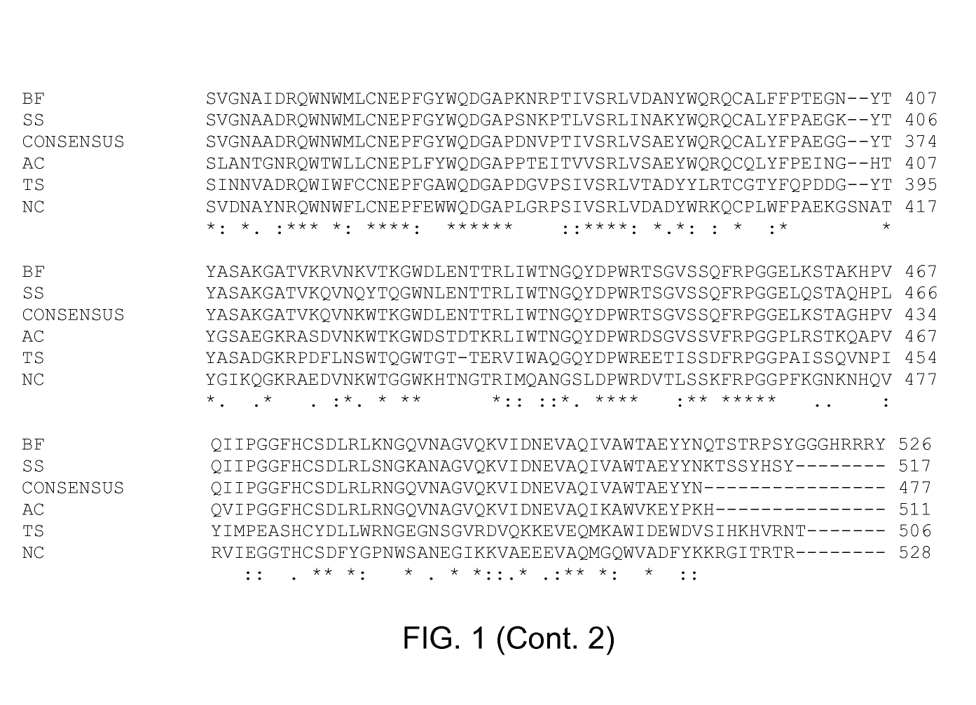

In some embodiments, a protease of the invention is any protease other than a prolyl endopeptidase from Aspergillus niger, defined by a consensus sequence based on multiple alignments of several homologs from various organisms, as provided in FIG. 1. The multiple sequence alignment shown in FIG. 1 was generated using ClustalW2, a general purpose multiple sequence alignment program, where the consensus sequence is marked as such.

The amino acid sequence of a naturally occurring protease can be altered in various ways known in the art to generate targeted changes in sequence and so provide variant sequences of the invention. Such variants will typically be functionally-preserved variants, which differ, usually in sequence, from the corresponding native or parent protein but still retain the desired or exhibit enhanced biological activity and/or function. Various methods known in the art can be used to generate targeted changes, e.g. phage display in combination with random and targeted mutations, introduction of scanning mutations, and the like, and provide a variant sequence of the invention. Included are the addition of His or epitope tags to aid in purification, as exemplified herein. Enzymes modified to provide for a specific characteristic of interest may be further modified, for e.g. by mutagenesis, exon shuffling, etc., as known in the art, followed by screening or selection, so as to optimize or restore the activity of the enzyme, e.g. to wild-type levels, and so provide other variant sequences of the invention.

The term “protease” also includes biologically active fragments. Fragments of interest include fragments of at least about 20 contiguous amino acids, more usually at least about 50 contiguous amino acids, and may comprise 100 or more amino acids, up to the complete protein, and may extend further to comprise additional sequences. In each case, the key criterion is whether the fragment retains the ability to digest gluten oligopeptides.

Modifications of interest to the protease that do not alter primary sequence but provide other variant proteases of the invention include chemical derivatization of proteins, including, for example, acylation with, e.g. lauryl, stearyl, myrsityl, decyl, or other groups; PEGylation, esterification; and/or amidation. Such modifications may be used to increase the resistance of the enzyme toward proteolysis, e.g. by attachment of PEG sidechains or lauryl groups to surface lysines. Also included are modifications of glycosylation, e.g. those made by modifying the glycosylation patterns of a protein during its synthesis and processing or in further processing steps; e.g. by exposing the protein to enzymes that affect glycosylation, such as glycosylating or deglycosylating enzymes. Also embraced are sequences that have phosphorylated amino acid residues, e.g. phosphotyrosine, phosphoserine, or phosphothreonine.

In some embodiments, a protease of the invention is subject to cleavage or removal of sequences that are not required for activity, including the removal of sequences to activate the protease, as in zymogen activation. Thus, a protease of the invention includes not only the “mature” or active form of a protease provided by the invention but also proteases with one or more additional amino acid sequences not required for activity (sometime referred to as “pre”, “pro”, or “prepro” forms of the protease, particularly where the naturally occurring protease is secreted from the producing cell as a zymogen). Zymogens are inactive forms of proteases that are converted to the active protease by proteolytic cleavage of a propeptide. In some embodiments of the methods of the invention, the protease of the invention is manufactured and dosed as a zymogen, and in these embodiments, the propeptide form is delivered and activated at the site of action (i.e., in the saliva or stomach) or preactivated prior to or contemporaneously with contact with a gluten-containing food. Thus, a zymogen form of a protease can be used to facilitate production or processing, and then, prior to use, be subjected to treatment such that the pro-peptide region of the zymogen is cleaved (and optionally purified away from the active protease). Such pre-activation of a zymogen form may be employed, e.g., to simplify the dosing formulation and/or to reduce the need for activation at the site of action. In other embodiments, the mature form of the enzyme is manufactured and dosed.

Also useful in the practice of and provided by the present invention are proteins that have been modified using molecular biological techniques and/or chemistry so as to improve their resistance to proteolytic degradation and/or to acidic conditions such as those found in the stomach, and to optimize solubility properties or to render them more suitable as a therapeutic agent. For example, the backbone of the protease can be cyclized to enhance stability (see Friedler et al. (2000) J. Biol. Chem. 275:23783-23789). Analogs of such proteins include those containing residues other than naturally occurring L-amino acids, e.g. D-amino acids or non-naturally occurring synthetic amino acids.

Thus, a protease of the invention includes proteases related in sequence and/or activity to a protease in Table 1, including but not limited to a recombinant or purified form of a protease having a kcat/Km of at least about 2.5 s⁻¹ M⁻¹, usually at least about 250 s⁻¹ M⁻¹ and preferably at least about 25000 s⁻¹ M⁻¹ for cleavage of a gluten oligopeptide that is immunogenic to a celiac disease patient, particularly of longer, physiologically generated peptides, for example the 33-mer from alpha-gliadin, (SEQ ID NO:25)LQLQPF(PQPQLPY)₃PQPQPF, and the 26-mer from gamma-gliadin, (SEQ ID NO:26) FLQPQQPFPQQPQQPYPQQPQQPFPQ.

A protease useful in the practice of the present invention can be characterized by its ability to cleave a pretreated substrate to remove toxic (“toxic” as used herein means capable of generating a harmful immune reaction in a celiac disease patient) gluten oligopeptides, where a “pretreated substrate” is a gliadin, hordein, secalin or avenin protein that has been treated with physiological quantities of gastric and pancreatic proteases, including pepsin (1:100 mass ratio), trypsin (1:100), chymotrypsin (1:100), elastase (1:500), and carboxypeptidases A and B (1:100), optionally in combination with another glutenase, as described below. Pepsin digestion may be performed at pH 2 for 20 min., to mimic gastric digestion, followed by further treatment of the reaction mixture with trypsin, chymotrypsin, elastase and carboxypeptidase at pH 7 for 1 hour, to mimic duodenal digestion by secreted pancreatic enzymes. The pretreated substrate comprises oligopeptides resistant to digestion, e.g. under physiological conditions. A glutenase may catalyze cleavage of pepsin-trypsin-chymotrypsin-elastase-carboxypeptidase (PTCEC) treated gluten such that less than 10% of the products are longer than PQPQLPYPQ (as judged by longer retention times on a C18 reverse phase HPLC column monitored at A₂₁₅). Glutenase assays suitable for characterizing proteases of the invention are also described in U.S. Pat. Nos. 7,303,871; 7,320,788; and 7,534,426, each of which is incorporated herein by reference.

The ability of a protease to cleave a pretreated substrate can be determined by measuring the ability of an enzyme to increase the concentration of free NH₂-termini in a reaction mixture containing 1 mg/ml pretreated substrate and 10 μg/ml of the protease, incubated at 37° C. for 1 hour. A protease useful in the practice of the present invention will increase the concentration of the free amino termini under such conditions, usually by at least about 25%, more usually by at least about 50%, and preferably by at least about 100%. A protease includes an enzyme capable of reducing the residual molar concentration of oligopeptides greater than about 1000 Da in a 1 mg/ml “pretreated substrate” after a 1 hour incubation with 10 μg/ml of the enzyme by at least about 2-fold, usually by at least about 5-fold, and preferably by at least about 10-fold. The concentration of such oligopeptides can be estimated by methods known in the art, for example size exclusion chromatography and the like.

A protease of the invention includes an enzyme capable of detoxification of whole gluten, as monitored by polyclonal T cell lines derived from intestinal biopsies of celiac patients; detoxification of whole gluten as monitored by LC-MS-MS; and/or detoxification of whole gluten as monitored by ELISA assays using monoclonal antibodies capable of recognizing sequences specific to gliadin, optionally when used in combination with a second glutenase. A protease of the invention may also include an enzyme that reduces the peripheral blood gluten-specific T cell response (see, for example, Anderson et al. (2005) Gut 54(9):1217-23, herein specifically incorporated by reference) to a “gluten challenge diet” in a celiac disease patient by at least about 2-fold, more usually by at least about 5-fold, and preferably by at least about 10-fold, optionally when used in combination with a second glutenase. A “gluten challenge diet” is defined as the intake of 100 g bread per day for 3 days by an adult celiac disease patient previously on a gluten-free diet. The peripheral blood gluten-specific T cell response can be measured in peripheral blood using standard clinical diagnostic procedures, as known in the art.

The proteases useful in the practice of the present invention may also be isolated and purified in accordance with conventional methods from recombinant production systems and from natural sources. Protease production can be achieved using established host-vector systems in organisms such as E. coli, S. cerevisiae, P. pastoris, Lactobacilli, Bacilli and Aspergilli. Integrative or self-replicative vectors may be used for this purpose. In some of these hosts, the protease is expressed as an intracellular protein and subsequently purified, whereas in other hosts the enzyme is secreted into the extracellular medium. Purification of the protein can be performed by a combination of ion exchange chromatography, Ni-affinity chromatography (or some alternative chromatographic procedure), hydrophobic interaction chromatography, and/or other purification techniques. Typically, the compositions used in the practice of the invention will comprise at least 20% by weight of the desired product, more usually at least about 50% by weight, preferably at least about 85% by weight, at least about 90%, and for therapeutic purposes, may be at least about 95% by weight, in relation to contaminants related to the method of preparation of the product and its purification. Usually, the percentages will be based upon total protein. Proteins in such compositions may be present at a concentration of at least about 500 μg/ml; at least about 1 mg/mg; at least about 5 mg/ml; at least about 10 mg/ml, or more. Suitable methods include those described in PCT Pub. No. 2008/115428, incorporated herein by reference.

In one aspect, the present invention provides a purified preparation of a protease. Such enzymes may be isolated from natural sources, but the present invention allows them to be produced by recombinant methods. In one embodiment, such methods utilize a fungal host for expression, although bacterial and eukaryotic systems, including insect systems, find use for some purposes. Coding sequences that contain a signal sequence, or that are engineered to contain a signal sequence, can be secreted into the media by the fungal host.

Where the enzyme is a cytoplasmic enzyme, a signal sequence can be introduced for periplasmic secretion, or the enzyme can be isolated from a cytoplasmic lysate. Methods for purification include Ni-NTA affinity purification, e.g. in combination with introduction of a histidine tag; and chromatography methods known in the art, e.g. cation exchange, anion exchange, gel filtration, HPLC, FPLC, and the like.

For various purposes, such as stable storage, the enzyme may be lyophilized. Lyophilization is preferably performed on an initially concentrated preparation, e.g. of at least about 1 mg/ml. Peg may be added to improve the enzyme stability. It has been found that MX PEP can be lyophilized without loss of specific activity. The lyophilized enzyme and excipients is useful in the production of enteric-coated capsules or tablets, e.g. a single capsule or tablet may contain at least about 1 mg. enzyme, usually at least about 10 mg enzyme, and may contain at least 100 mg enzyme, at least about 500 mg enzyme, or more. Coatings may be applied, where a substantial fraction of the activity is retained, and is stable for at least about 1 month at 4° C.

For purposes of combinations of enzymes, the following non-limiting list of proteases is of interest: Hordeum vulgare endoprotease (Genbank accession U19384); aspergillopepsin from Aspergillus Niger (GenBank ID#CAK42031); X-Pro dipeptidase from Aspergillus oryzae (GenBank ID#BD191984); carboxypeptidase from Aspergillus saitoi (GenBank ID#D25288); Flavobacterium meningosepticum PEP (Genbank ID #D10980); Sphingomonas capsulata PEP (Genbank ID#AB010298); Penicillium citrinum PEP (Genbank ID#D25535); Lactobacillus helveticus PEP (Genbank ID#321529); and Myxococcus xanthus PEP (Genbank ID#AF127082). In other embodiments a protease of the invention may be combined with Hordeum vulgare endopeptidase B (EPB2), and the like. By combination, it is intended that a plurality of proteases are administered contemporaneously in separate formulations, or are co-formulated. In some embodiments the protease or combination of proteases is ingested by an individual contemporaneously with food, e.g. at meal time. The proline- and glutamine-specific proteases described in U.S. Pat. Nos. 7,303,871 and 7,320,788 and in PCT Pub. Nos. 2010/047733, 2009/075816, and 2008/115411, each of which is incorporated herein by reference are especially suitable for use in such combinations.

The proteases can be combined, in accordance with the present invention, with glutamine-specific proteases, such as the barley EPB2 protease or its recombinant form ALV001, to make highly potent, gluten-degrading mixtures of proteases.

The methods of the invention, as well as tests to determine their efficacy in a particular patient or application, can be carried out in accordance with the teachings herein using procedures standard in the art. Thus, the practice of the present invention may employ conventional techniques of molecular biology (including recombinant techniques), microbiology, cell biology, biochemistry and immunology within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction” (Mullis et al., eds., 1994); and “Current Protocols in Immunology” (J. E. Coligan et al., eds., 1991); as well as updated or revised editions of all of the foregoing.

For the purposes of the present invention, immunogenic gliadin oligopeptides are peptides derived during normal human digestion of gliadins and related storage proteins from dietary cereals, e.g. wheat, rye, barley, and the like, that are immunogenic in celiac disease patients, e.g., act as antigens for T cells. Immunogenic peptides are usually from about 8 to 20 amino acids in length, more usually from about 10 to 18 amino acids or longer. Such peptides may include PXP motifs. Determination of whether an oligopeptide is immunogenic for a particular patient is readily determined by standard T cell activation and other assays known to those of skill in the art. Determination of whether a candidate enzyme will digest a toxic gluten oligopeptide can be empirically determined. For example, a candidate may be combined with an oligopeptide or with a pretreated substrate comprising one or more of gliadin, hordein, secalin or avenin proteins that have been treated with physiological quantities of gastric and pancreatic proteases. In each instance, it is determined whether the enzyme is capable of cleaving the oligopeptide. The oligopeptide or protein substrates for such assays may be prepared in accordance with conventional techniques, such as synthesis, recombinant techniques, isolation from natural sources, or the like. For example, solid-phase peptide synthesis involves the successive addition of amino acids to create a linear peptide chain (see Merrifield (1963) J. Am. Chem. Soc. 85:2149-2154). Recombinant DNA technology can also be used to produce the peptide.

The level of digestion of the toxic oligopeptide can be compared to a baseline value. Gluten becomes much less toxic when it is degraded to peptides shorter than 10 amino acids in length, such as peptides of 8 amino acids, peptides of 6 amino acids, or shorter peptides. The disappearance of the starting material and/or the presence of digestion products can be monitored by conventional methods in model systems, including in vitro and in vivo assay systems. For example, a detectable marker can be conjugated to a peptide, and the change in molecular weight associated with the marker is then determined, e.g. acid precipitation, molecular weight exclusion, and the like. The baseline value can be a value for a control sample or a statistical value that is representative a control population. Various controls can be conducted to ensure that an observed activity is authentic, including running parallel reactions, positive and negative controls, dose response, and the like.

The present invention also provides recombinant nucleic acids comprising coding sequences for the recombinant proteases of the invention. These recombinant nucleic acids include those with nucleotide sequences comprising one or more codons optimized for expression in Pichia pastoris, E. coli, or other host cells heterologous to the cells in which such proteins (or their variants) are naturally produced. Examples of optimized nucleotide sequences are provided in the sequence listing as SEQ ID NO:13-24.

The present invention also provides recombinant expressing vectors comprising nucleic acids encoding the proteases of the invention operably linked to a promoter positioned to drive expression of the coding sequence in a host cell. The present invention also provides methods for producing the proteases of the invention comprising culturing a host cell comprising an expression vector of the invention under conditions suitable for expression of the protease.

As used herein, compounds which are “commercially available” may be obtained from commercial sources including but not limited to Acros Organics (Pittsburgh Pa.), Aldrich Chemical (Milwaukee Wis., including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), Fisher Scientific Co. (Pittsburgh Pa.), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.), Maybridge Chemical Co. Ltd. (Cornwall U.K.), Parish Chemical Co. (Orem Utah), Pfaltz & Bauer, Inc. (Waterbury Conn.), Polyorganix (Houston Tex.), Pierce Chemical Co. (Rockford Ill.), Riedel de Haen AG (Hannover, Germany), Spectrum Quality Product, Inc. (New Brunswick, N.J.), TCI America (Portland Oreg.), Trans World Chemicals, Inc. (Rockville Md.), Wako Chemicals USA, Inc. (Richmond Va.), Novabiochem and Argonaut Technology.

### Formulations

The present invention arose in part from the discovery that certain PEP enzymes are buffer-dependent with respect to their ability to digest intact gluten, while other PEP enzymes are unable to digest intact gluten in any of the buffers tested. In particular, carboxylic acid compounds such as citric acid, citrate, acetate or acetic acid can enhance the activity of these enzymes and allow them to proteolyze intact gluten proteins. Thus, the ability of certain members of the S28 family of proteases (lysosomal Pro-Xaa carboxypeptidases) to cleave intact gluten proteins are dependent upon the addition of buffers, including but not limited to acetate and citrate. However, even for those enzymes that are able to digest intact gluten in the presence of buffer, the concentration of buffer necessary to allow the proteases to degrade intact gluten is relatively high and difficult to achieve in vivo. Therefore, the present invention provides for the use of combinations of enzymes for efficient degradation of intact gluten, including degradation in vivo.

Enzymes including AC PEP, MG PEP, NC PEP, TS PEP, and the insect S28 family protease have not been found to degrade intact gluten proteins even in the presence of up to 400 mM citrate or acetate buffer.

In some embodiments, the invention provides compositions of enzymes that are S28 family (lysosomal Pro-Xaa carboxypeptidase) proteases in combination with carboxylic acid compound buffers, which enhance the protease activity of the enzyme and allow them to proteolyze intact gluten proteins. Specific proteases that benefit from such a buffer formulation include AN PEP (a prolyl endopeptidase from Aspergillus niger, which is the protease in brewers clarex; see U.S. Pat. Nos. 7,323,327 and 7,309,595), BF PEP, and SS PEP. Effective concentrations of the buffering agent range from about 200 mM to about 400 mM. At buffer concentrations of greater than about 200 mM, the enzymes are able to degrade intact gluten, although in the absence of buffer these enzymes have minimal activity on intact gluten proteins. This buffer dependence may reflect an interaction between the buffer and the enzyme that changes the enzyme substrate specificity. These enzymes are able to degrade smaller gluten peptides even in the absence of buffer, and can therefore be used in accordance with the invention for degradation of gluten when combined with one or more additional protease enzymes that cleave intact gluten into smaller gluten peptides.

Thus, in other embodiments, the invention provides a formulation comprising a combination of an S28 family (lysosomal Pro-Xaa carboxypeptidase) protease with a second gluten cleaving protease, particularly a second gluten cleaving protease having a specificity other than that of the S28 family protease that is able to degrade intact gluten proteins into smaller peptide fragments that are substrates for the S28 family protease.

Such combinations of enzymes include combinations of one or more of the proteases of the invention with one or more of the following non-limiting list of proteases: Hordeum vulgare endoprotease B (Genbank accession U19384); Hordeum vulgare endoprotease A (Genbank accession CAB09697.1); X-Pro dipeptidase from Aspergillus oryzae (GenBank ID#BD191984); carboxypeptidase from Aspergillus saitoi (GenBank ID#D25288); and aspergillopepsin from Aspergillus niger (GenBank ID#EHA27889). Combinations of interest include, without limitation, a combination of a protease disclosed herein with a proline specific protease (see, e.g., PCT Pat. Pub. No. 2011/126873, incorporated herein by reference), a combination with a glutamine specific cysteine protease (see, for example US Patent publication no. 2012/031502, incorporated herein by reference; Hordeum vulgare endoprotease (Genbank accession U19384; wheat cysteine protease Yang et al. (2011) J Sci Food Agric. 91(13):2437-42; or other plant cysteine proteases as reviewed by Grudkowska and Zagdanska (2004) Acta Biochimica Polonica 51(3), incorporated herein by reference), a combination with any grain derived cysteine protease or insect protease, including insect cysteine endoprotease, and/or a combination with any grain derived aspartic protease, mammal derived aspartic protease, or fungal aspartic protease including aspergillopepsin. For other suitable buffers and excipients, see PCT Pub. Nos. 2010/021752 and 2010/042203, incorporated herein by reference. Combinations include for example and without limitation, a protease set forth herein with any protease described in U.S. Pat. Nos. 7,320,788 and 7,628,985.

By combination, it is intended that a plurality of proteases are administered contemporaneously in separate formulations, or are co-formulated. In some embodiments the protease or combination of proteases is ingested by an individual contemporaneously with food, e.g. at meal time or at any other time when a food is ingested. The proline- and glutamine-specific proteases described in U.S. Pat. Nos. 7,303,871 and 7,320,788 and in PCT Pub. Nos. 2010/047733, 2009/075816, and 2008/115411, each of which is incorporated herein by reference are especially suitable for use in such combinations. A preferred glutamine-specific protease for use in the combination protease formulations of the invention is EP-B2, derived from barley (see U.S. Pat. No. 7,320,788 and U.S. Patent Application Pub. No. US2010/0092451). Other proteases useful for such purposes include those described in US2011/0236369; US2011/0171201; US 2005/0064403; US 2009/0275079; U.S. Pat. No. 7,323,327; WO 2002/068623; and WO 2002/068623.

In some embodiments, a formulation comprising a combination of proteases, for example a cysteine endoprotease such as EP-B2, capable of degrading intact gluten proteins into gluten peptides, and a PEP as described herein, is capable of degrading gluten into oligopeptides of less than 8 amino acids in length, and may be given in a unit dose of between 1-1000 mg of the cysteine endoprotease and between 1-1000 mg of the PEP administered either together or separately. The weight ratio of the two enzymes can be optimized based upon the ability of the combination to degrade the immunogenic portions of gluten in vitro or in vivo. Useful cysteine protease:PEP weight ratios include between 1:50 to 50:1, more preferably between 1:20 to 20:1, and most preferably between 1:5 to 5:1. Typical dosage forms of the combination protease include a tablet, capsule, or sachet containing between 10-300 mg of each protease. For example, a 100 mg unit dose of combination protease may be packaged in a sachet that is either reconstituted in a drink or sprinkled on the food item.

The protease combination may be administered together with buffering excipients such as citric acid, sodium citrate, potassium citrate, sodium acetate, calcium carbonate, malic acid, tartaric acid, sodium tartrate, potassium tartrate, sodium phosphate, potassium phosphate, sodium lactate, and/or potassium lactate. The listed buffering excipients may be used in the orally administered combination protease formulation to maintain the stomach at a desired pH to enhance the stability and/or activity of the combination protease by using the appropriate ratios and amounts of the buffering excipients. The buffering excipients may also be used to control the pH of a solid or liquid dosage form in order to improve combination protease stability prior to, during, and/or following administration to the patient. Typically, the buffering excipient will contain at least one acid and one base, usually as a sodium, calcium, or potassium salt, used in combination at a ratio that results in the desired pH. Suitable pH ranges are between pH 2-7, more typically pH 3-6, and most typically between pH 4-5.5. In addition, the use of citric acid, sodium citrate, potassium citrate, and sodium acetate may be used to enhance degradation of gluten proteins by modifying the substrate specificity of the PEPs as described herein.

Compounds useful for co-administration with the proteases and treated foodstuffs of the invention can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services.

The proteases of the invention and/or the compounds and combinations of enzymes administered therewith are incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the protease and/or other compounds can be achieved in various ways, usually by oral administration. The protease and/or other compounds may be systemic after administration or may be localized by virtue of the formulation, or by the use of an implant that acts to retain the active dose at the site of implantation.

In pharmaceutical dosage forms, the protease and/or other compounds may be administered in the form of their pharmaceutically acceptable salts, or they may also be used alone or in appropriate association, as well as in combination with other pharmaceutically active compounds. The agents may be combined, as previously described, to provide a cocktail of proteolytic activities. The following methods and excipients are exemplary and are not to be construed as limiting the invention.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Gluten detoxification for a gluten sensitive individual can commence as soon as food enters the stomach, because the acidic environment (˜pH 2-4) of the stomach favors gluten solubilization. Introduction of a combination of a protease of the invention, especially in combination with another glutenase, into the stomach can, in combination with the action of pepsin and other stomach enzymes and conditions, lead to accelerated destruction of toxic peptides upon entry of gluten in the small intestines of celiac patients. Such proteases may not require enteric formulation.

In another embodiment, the protease is admixed with food, or used to pre-treat foodstuffs containing glutens. Protease mixed in foods can be enzymatically active prior to or during ingestion, and may be encapsulated or otherwise treated to control the timing of activity. Alternatively, the protease may be encapsulated to achieve a timed release after ingestion, e.g. a predetermined period of time after ingestion and/or a predetermined location in the intestinal tract.

Formulations are typically provided in a unit dosage form, where the term “unit dosage form,” refers to physically discrete units suitable as unitary dosages for human subjects, each unit containing a predetermined quantity of protease in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Depending on the patient and condition being treated and on the administration route, the protease may be administered in dosages of 0.01 mg to 500 mg/kg body weight per day, e.g. about 1-100 mg/kg body weight/per day, e.g., 20 mg/kg body weight/day for an average person. Efficient proteolysis of gluten in vivo for an adult may require at least about 500 units of a therapeutically efficacious enzyme, or at least about 5000 units, or at least about 50,000 units, at least about 500,000 units, or more, for example, about 5×10⁶ units or more, where one unit is defined as the amount of enzyme required to hydrolyze 1 μmol of a chosen substrate per min under specified conditions. It will be understood by those of skill in the art that the dose can be raised, but that additional benefits may not be obtained by exceeding the useful dosage. Those of skill in the art will appreciate that the orally administered proteases of the invention are non-toxic, so the amount of protease administered can exceed the dose sufficient to degrade a substantial amount (e.g., 50% or more, such as 90% or 99%) or all of the gluten in the food with which it is consumed. Dosages will be appropriately adjusted for pediatric formulation. In children the effective dose may be lower. In combination therapy, a comparable dose of the two enzymes may be given; however, the ratio may be influenced by e.g., synergy in activity and/or the relative stability of the two enzymes toward gastric and duodenal inactivation.

Protease treatment of celiac disease or other form of gluten intolerance is expected to be most efficacious when administered before or with meals. However, since food can reside in the stomach for 0.5-2 h, the protease could also be administered up to within 1 hour after a meal. In some embodiments of the invention, formulations comprise a cocktail of selected proteases, for example a combination of a protease of the invention with one or more of Sphingomonas capsulata PEP, Hordeum vulgare cysteine endoprotease B (EPB2), and the like. Such combinations may achieve a greater therapeutic efficacy.

Those of skill will readily appreciate that dose levels can vary as a function of the specific enzyme, the severity of the symptoms and the susceptibility of the subject to side effects. Some of the proteases are more potent than others. Preferred dosages for a given enzyme are readily determinable by those of skill in the art by a variety of means. A preferred means is to measure the physiological potency of a given compound.

The compositions of the invention can be used for prophylactic as well as therapeutic purposes. As used herein, the term “treating” refers both to the prevention of disease and the treatment of a disease or a pre-existing condition and more generally refers to the prevention of gluten ingestion from having a toxic effect on the patient or reducing the toxicity, relative to the toxic effect of ingestion of the same amount of gluten in the absence of protease therapy. The invention provides a significant advance in the treatment of ongoing disease, and helps to stabilize and/or improve the clinical symptoms of the patient. Such treatment is desirably performed prior to loss of function in the affected tissues but can also help to restore lost function or prevent further loss of function. Evidence of therapeutic effect may be any diminution in the severity of disease, particularly as measured by the severity of symptoms such as fatigue, chronic diarrhea, malabsorption of nutrients, weight loss, abdominal distension, anemia, skin rash, and other symptoms of celiac disease and/or dermatitis herpetiformis and/or gluten sensitivity. Other disease indicia include the presence of antibodies specific for glutens, the presence of antibodies specific for tissue transglutaminase, the presence of pro-inflammatory T cells and cytokines, damage to the villus structure of the small intestine as evidenced by histological or other examination, enhanced intestinal permeability, and the like.

Patients that may be treated by the methods of the invention include those diagnosed with celiac disease or other gluten intolerance through one or more of serological tests, e.g. anti-gliadin antibodies, anti-transglutaminase antibodies, anti-endomysial antibodies; endoscopic evaluation, e.g. to identify celiac lesions; histological assessment of small intestinal mucosa, e.g. to detect villous atrophy, crypt hyperplasia, infiltration of intra-epithelial lymphocytes; and any GI symptoms dependent on inclusion of gluten in the diet.

Given the safety of oral proteases, they also find a prophylactic use in high-risk populations, such as Type I diabetics, family members of diagnosed celiac disease patients, dermatitis herpetiformis patients, HLA-DQ2 positive individuals, and/or patients with gluten-associated symptoms that have not yet undergone formal diagnosis. Such patients may be treated with regular-dose or low-dose (10-50% of the regular dose) enzyme. Similarly, temporary high-dose use of such an agent is also anticipated for patients recovering from gluten-mediated enteropathy in whom gut function has not yet returned to normal, for example as judged by fecal fat excretion assays.

Patients that can benefit from the present invention may be of any age and include adults and children. Children in particular benefit from prophylactic treatment, as prevention of early exposure to toxic gluten peptides can prevent initial development of the disease. Children suitable for prophylaxis can be identified by genetic testing for predisposition, e.g. by HLA typing, by family history, by T cell assay, or by other medical means. As is known in the art, dosages may be adjusted for pediatric use.

The therapeutic effect can be measured in terms of clinical outcome or can be determined by immunological or biochemical tests. Suppression of the deleterious T-cell activity can be measured by enumeration of reactive Th1 cells, by quantitating the release of cytokines at the sites of lesions, or using other assays for the presence of autoimmune T cells known in the art. Alternatively, one can look for a reduction in symptoms of a disease.

Various methods for administration may be employed, preferably using oral administration, for example with meals. The dosage of the therapeutic formulation will vary widely, depending upon the nature of the disease, the frequency of administration, the manner of administration, the clearance of the agent from the host, and the like. The initial dose can be larger, followed by smaller maintenance doses. The dose can be administered as infrequently as weekly or biweekly, or more often fractionated into smaller doses and administered daily, with meals, semi-weekly, or otherwise as needed to maintain an effective dosage level.

The various aspects and embodiments of the invention are illustrated without limitation in the following examples.

### EXAMPLES

The following examples are put forth so as to provide those of ordinary skill in the art with a complete disclosure and description of how to make and use the present invention, and are not intended to limit the scope of the invention or to represent that the experiments below are all or the only experiments performed. Efforts have been made to ensure accuracy with respect to numbers used (e.g., amounts, temperature, and the like), but some experimental errors and deviations may be present. Unless indicated otherwise, parts are parts by weight, molecular weight is weight average molecular weight, temperature is in degrees Centigrade, and pressure is at or near atmospheric.

Cloning and Expression of Proline Specific Endopeptidases in Pichia pastoris:

Codon optimized nucleotide sequences (SEQ ID NO: 13-16, 21-24) were synthesized and cloned into pPINKα-HC vector (Invitrogen) with the α-mating factor sequence appended to the N-terminus of each protease for secreted expression in Pichia pastoris strains 1 and/or 4 using the PichiaPINK® kit (Invitrogen). The α-mating factor secretion signal may be removed upon secretion of the protein. Electrocompetent P. pastoris cells were prepared and transformed with the expression plasmids. The strains in the PichiaPink kits are ade2 auxotrophs. The expression plasmids contains a copy of the ADE2 gene which complements the adenine auxotrophy. Transformation of the PichiaPink strains with the expression plasmids enables the strain to grow on medium lacking adenine (Ade dropout medium). The transformants were selected on Ade dropout plates and screened for expression of the proteases.

For protein expression, a 10 mL starter culture was grown for 14 hours in Buffered Glycerol-complex Medium (BMGY) in a 50 mL conical tube at 24-28° C. The starter culture was used to inoculate 500 mL of BMGY in a 2 L shake flask. Cells were grown for 48 hours at 24-28° C. while shaking at 250 rpm. Cells were centrifuged and resuspended in 100 mL of Buffered Methanol-complex Medium (BMMY) containing 0.5% methanol to induce protein expression under the control of methanol inducible AOX1 promoter. For proteins MG and TS PEP, the cells were resuspended in 50 mL Buffered Minimal Media (BMM) containing 100 mM phosphate buffer pH 6.0 and 0.5% methanol. Protein was expressed for 48 hours at 24-28° C. while shaking at 250 rpm with 0.5% methanol supplementation after 8 and 24 hours.

Purification of Proline Specific Endopeptidases from Pichia pastoris:

After expression of the protein, the cells were removed by centrifugation. The supernatant was filtered through a 0.22 um PVDF syringe filter and combined 1:1 (v:v) with 400 mM acetate buffer, pH 4.0. The supernatant was loaded onto a 5 ml HiTrap SP FF column, and 50 mM acetate, pH 4.0 buffer was used as buffer A. Endopeptidase XP_{—}001273182 (AC PEP) and XP_{—}382380 (GZ PEP) were washed with 0 mM and 300 mM NaCl buffer and eluted with 800 mM NaCl buffer. Endopeptidase XP_{—}001551952 (BF PEP) was washed with 0 mM NaCl buffer and eluted with 400 mM NaCl buffer. NC PEP was washed with 300 mM NaCl and eluted with 600 mM NaCl. SS PEP supernatant was diluted an additional 2-fold with water before loading onto the SP column. SS PEP was eluted with 400 mM NaCl in buffer A. MG and TS PEP did not bind to the SP column and were concentrated using 10,000 MWCO Amicon ultrafiltration spin column. HiTrap SP FF purified endopeptidases were further concentrated using an ultrafiltration spin column with 10,000 MWCO. Endopeptidases were then flash frozen and stored below −70 deg C.

Cloning and Expression of Proline Specific Endopeptidases in Escherichia coli (E. Coli):

Codon optimized nucleotide sequences (SEQ ID NO: 17-20) were synthesized and cloned into pET28b vector (Novagen) between NcoI and BamHI sites for the cytosolic expression in E. coli strain BL21 (DE3). Electrocompetent cells were transformed with expression plasmid. The expression plasmids contained the kan+gene to provide resistance to the antibiotic kanamycin. Transformation of the E. coli strains with the expression plasmids enabled the strain to grow on medium containing kanamycin. The transformants were selected on kanamycin containing plates and screened for expression of the proteases.

For protein expression, a 10 mL starter culture was grown for 12 hours in Luria Broth (LB) containing 50 ug/ml kanamycin in 15 ml test tubes at 37° C. with shaking at 250 rpm. The starter culture was used to inoculate 1000 mL of LB containing 50 ug/ml kanamycin in a 2 L shake flask. Cells were grown at 37° C. with shaking at 250 rpm to an optical density (OD₆₀₀) of 0.6-0.8. Isopropyl β-D-1-thiogalactopyranoside (IPTG) was added to a concentration of 0.4 mM to induce protein expression under the control of IPTG inducible T7 promoter. Protein was expressed for 4 hours at 37° C. with shaking at 250 rpm. Proteases expressed well in this expression system as inclusion bodies (IB).

Stability of Prolyl Endopeptidases to Pepsin and EP-B2 Under Acidic Conditions.

PEP from Aspergillus clavatus (AC PEP) was incubated with EP-B2 (450 ug/ml) or pepsin (100 ug/ml) at 37 deg C. for 10 minutes or 30 minutes in 300 mM citrate pH 3.3, acetate pH 4.0, and acetate pH 4.5. Samples were run on SDS PAGE to determine the level of enzyme degradation. The results demonstrate that AC PEP is resistant to EP-B2 and pepsin degradation over the pH range tested (pH 3.3-4.5).

pH Stability of Prolyl Endopeptidases:

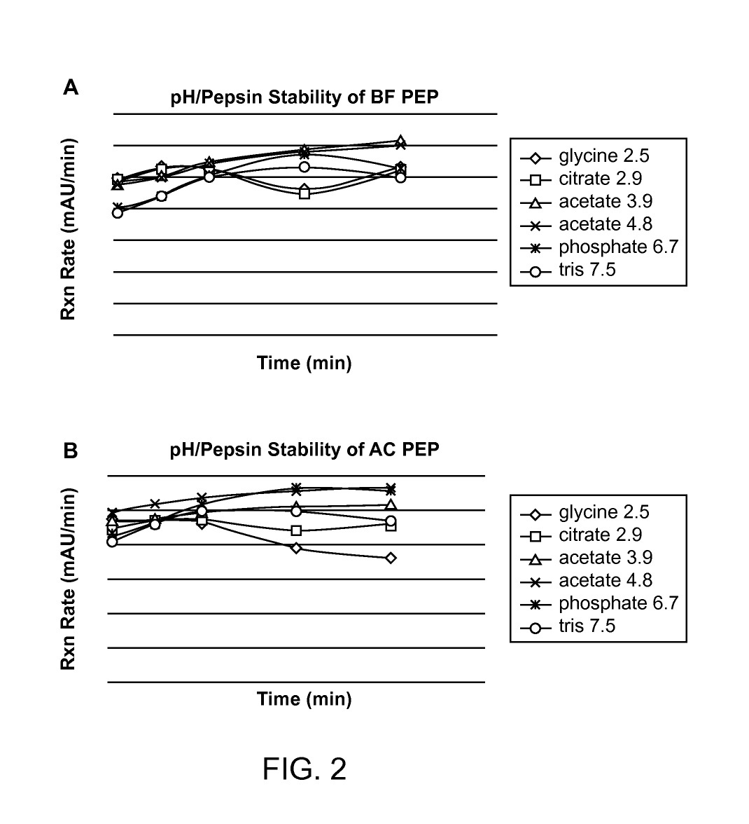

(A) 0.15 mg/ml prolyl endopeptidase from B. fuckeliana (BF PEP) or (B) 0.07 ring/rd prolyl endopeptidase from A. clavatus (AC PEP) were incubated at 37 degC in 200 mM buffers containing 1 mg/ml pepsin. At the indicated timepoints, samples were taken and placed in activity buffer (400 mM citrate pH 4.0±200 uM AFP-pNA substrate) to measure enzyme activity. The data, shown in FIG. 2A-2B, indicate that BF PEP and AC PEP have good stability over a wide pH range.

Activity of Prolyl Endopeptidases on Gluten.

20 ring/rd whole wheat bread gluten was degraded with enzymes for 30 minutes at 37° C. in a vegetable korma meal. Gluten degradation was measured using T cell lines generated from small intestinal biopsies of celiac disease patients. The combination of EP-B2 with either AC PEP or BF PEP results in significantly improved gluten degradation compared to EP-B2 alone.

| | Gluten | Gluten |
|---|---|---|
| | Degradation | Degradation |
| | (fold change | (fold change |
| | using T | using T |
| | Cell line #1) | cell line #2) |
| 0.15 mg/ml EP-B2 | 1.4 | 3.2 |
| 0.15 mg/ml EP-B2 + 0.15 mg/ml AC PEP | 6.7 | 18.2 |
| 0.15 mg/ml EP-B2 + 0.15 mg/ml BF PEP | 3.7 | 13.3 |
| 0.3 mg/ml EP-B2 | 2.3 | 12.5 |
| 0.3 mg/ml EP-B2 + 0.15 mg/ml AC PEP | 20 | 200 |
| 0.3 mg/ml EP-B2 + 0.15 mg/ml BF PEP | 9.1 | 67 |
| 0.6 mg/ml EP-B2 | 6.3 | 100 |

Ratio of EP-B2 to Prolyl Endopeptidase.

20 mg/ml gluten was degraded in 100 mM acetate pH 4.0 buffer using combinations of EP-B2 and AC PEP with the total enzyme concentration fixed at 0.3 mg/ml. Samples were analyzed using intestinal T cell lines obtained from celiac disease patients. Although AC PEP has minimal activity by itself, it significantly enhances degradation of gluten compared to EP-B2 alone at all ratios tested.

| | Gluten Degradation |
|---|---|
| Sample | (Fold-change) |
| Placebo | 1.0 |
| 0.3 mg/ml EP-B2 | 3.6 |
| 0.24 mg/ml EP-B2 + 0.06 mg/ml AC PEP | 36 |
| 0.15 mg/ml EP-B2 + 0.15 mg/ml AC PEP | 54 |
| 0.06 mg/ml EP-B2 + 0.24 mg/ml AC PEP | 25 |
| 0.6 mg/ml AC PEP | 1.7 |

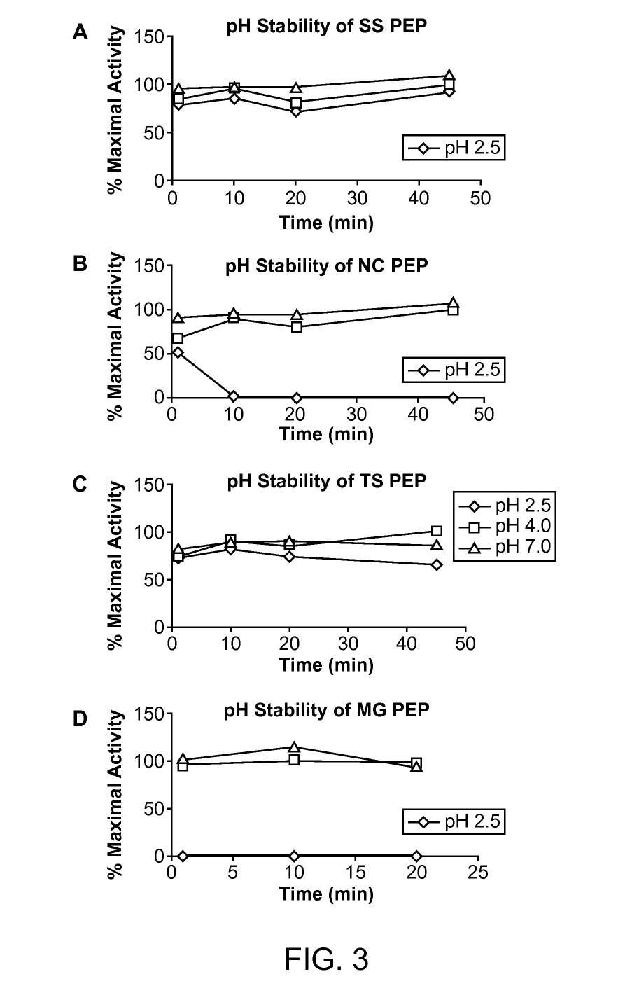

pH Stability of SS, MG, NC, and TS PEP. Approximately 0.5 mg/ml SS, NC, and TS PEP and 0.9 mg/ml MG PEP were incubated in 200 mM buffers with different pHs. At the indicated timepoint, samples were taken and added to activity buffer (400 mM acetate pH 4.0 with 200 uM AFP-pNA substrate) to measure enzyme activity. The results, shown in FIG. 3A-3D, indicate that all proteases are stable at pH 4.0 and 7.0 and that SS and TS PEP are stable at pH 2.5.

All publications, patents, and patent applications cited in this specification are herein incorporated by reference as if each individual publication, patent, or patent application were specifically and individually indicated to be incorporated by reference.

The present invention has been described in terms of particular embodiments found or proposed by the inventor to comprise preferred modes for the practice of the invention. It will be appreciated by those of skill in the art that, in light of the present disclosure, numerous modifications and changes can be made in the particular embodiments exemplified without departing from the intended scope of the invention. Moreover, due to biological functional equivalency considerations, changes can be made in methods, structures, and compounds without affecting the biological action in kind or amount. All such modifications are intended to be included within the scope of the appended claims.

1. A proline specific endopeptidase with at least 70% sequence identity to one of SEQ ID:1-12, in isolated, recombinant or purified form.

2. The protease of claim 1, wherein the protease has at least 90% sequence identity to one of SEQ ID:1-12.

3. The protease of claim 1, wherein the protease has at least 95% sequence identity one of SEQ ID:1-12.

4. The protease of claim 1, wherein said protease digests gluten fragments that are resistant to normal digestive enzymes.

5. The protease of claim 1, wherein said protease are formulated with a pharmaceutically acceptable excipient.

6. The protease of claim 1, wherein said protease is admixed with food.

7. The protease according to claim 1, wherein said protease is stable to acid conditions.

8. The protease of claim 1, wherein the protease can degrade gluten to fragments shorter than 8 amino acids in the presence of a carboxylic acid compound buffer at a concentration of at least 200 mM or in combination with another gluten cleaving protease.

9. A formulation comprising a proline specific endopeptidase according to claim 1, and one or more of citric acid, sodium acetate, sodium citrate, or excipient that contains a carboxylic acid moiety at a concentration of at least about 200 mM.

10. A formulation comprising the protease of claim 1, in combination with at least one other gluten cleaving protease and a pharmaceutically acceptable excipient.

11. The formulation of claim 10, wherein the gluten cleaving protease has a specificity other than that of the S28 family protease.

12. The formulation of claim 11, wherein the second gluten-cleaving protease is a cysteine endoprotease.

13. The formulation of claim 12, wherein the cysteine endoprotease is cysteine endoprotease B, isoform 2 from barley (EP-B2) (Genbank accession U19384).

14. The formulation of claim 12, wherein the cysteine endoprotease is a homolog, ortholog or variant of cysteine endoprotease B, isoform 2 from barley (EP-B2) (Genbank accession U19384).

15. The formulation of claim 12, wherein the cysteine endoprotease is a cysteine endoprotease from barley.

16. The formulation of claim 12, wherein the cysteine endoprotease is a cysteine endoprotease from a germinating grain.

17. The formulation of claim 12, wherein the cysteine endoprotease is an insect enzyme.

18. The formulation of claim 9, and further comprising aspergillopepsin I from Aspergillus niger (Genbank ID#EHA27889).

19. The formulation of claim 10, wherein the second gluten-cleaving protease is one or more of Hordeum vulgare endoprotease B (Genbank accession U19384); Hordeum vulgare endoprotease A (Genbank accession CAB09697.1); X-Pro dipeptidase from Aspergillus oryzae (GenBank ID#BD191984); carboxypeptidase from Aspergillus saitoi (GenBank ID#D25288) and aspergillopepsin from Aspergillus niger (GenBank ID#EHA27889).

20. A recombinant expression vector comprising a coding sequence for a protease, wherein said protease has at least 70% sequence identity to one of SEQ ID:1-12 and a promoter that drives expression of said protease in a suitable host cell.

21. A method for degrading gluten in food, said method comprising contacting gluten-containing food with a protease of claim 1.

22. A method for treating gluten intolerance, celiac disease, dermatitis herpetiformis and/or gluten sensitivity in a patient in need of such treatment, wherein said treatment reduces exposure of said patient to immunogenic gluten peptides, said method comprising the step of orally administering to said patient a therapeutically effective dose of a protease of claim 1 contemporaneously with the ingestion of a food that may contain gluten.

23. The method of claim 22, wherein said protease or formulation is administered in the form of a pharmaceutical formulation that comprises at least one pharmaceutically acceptable excipient.
